(19) United States
(12) Patent Application Publication
(10) Pub. No.: US 2020/0308591 A1
(43) Pub. Date: Oct. 1, 2020

(54) PROMOTER REGION ANALYSIS METHODS AND CELLS FOR PRACTICING SAME

(71) Applicant: Eurofins DiscoverX Corporation, Fremont, CA (US)

(72) Inventors: Paul Shapiro, Half Moon Bay, CA (US); Venkatesh Chari, Fremont, CA (US); Jennifer Lin-Jones, Castro Valley, CA (US); Jane Lamerdin, Livermore, CA (US)

(73) Assignee: Eurofins DiscoverX Corporation, Fremont, CA (US)

(21) Appl. No.: 16/831,586

(22) Filed: Mar. 26, 2020

Related U.S. Application Data
(60) Provisional application No. 62/825,559, filed on Mar. 28, 2019.

(51) Int. Cl. C12N 15/63; C12Q 1/34

(52) U.S. Cl. CPC C12N 15/63; C12Y 302/01023; C12Q 1/34

(57) ABSTRACT

Provided are methods of assessing activity of a promoter region. The methods include culturing a cell including a nucleic acid, the nucleic acid including a region that encodes an enzyme donor (ED) operably coupled to a promoter region, under conditions in which the ED is expressed when the promoter region is active. The methods further include contacting the ED, if expressed, with an enzyme acceptor (EA) to form ED-EA complexes having enzymatic activity. The methods further include detecting the level of the enzymatic activity to assess activity of the promoter region. Activity of the promoter region may be indicative, and therefore may be used to assess, the activity of a cellular signaling pathway of interest and/or of endogenous or exogenous (e.g., introduced) transcription factors of interest. Cells, compositions, and kits that find use, e.g., in practicing the methods of the present disclosure, are also provided.

### CROSS REFERENCE TO RELATED APPLICATION

This application claims priority to U.S. Provisional Application Ser. No. 62/825,559 filed Mar. 28, 2019, which is hereby incorporated by reference in its entirety.

### STATEMENT OF GOVERNMENTAL SUPPORT

NONE

### FIELD OF TECHNOLOGY

This invention is generally directed to methods of assessing activity of a promoter region, and more specifically, promoter region coupled to a detection agent such that expression of the promoter region directs expression of the detection agent measuring the activity of the promoter region. The disclosed method assess the activity of the cell signaling pathways and effect of a test compound on cell signaling pathways.

### REFERENCE TO SEQUENCE LISTING

The instant application contains a Sequence Listing which has been submitted as an ASCII text file and is hereby incorporated by reference in its entirety. This text file was created on Mar. 22, 2020 is named “PBH_010_1Seq_List.txt” and is 49,795 bytes in size.

### BACKGROUND

Interests in exploring various aspects of cell signaling pathways and effect of different compounds or molecules on the cell signaling pathway regulation has been a key driving force in understanding diseases and finding remedies. As more proteins in cell signaling pathways and their functions are identified, interest in finding molecules that modulate the activity of these proteins is growing tremendously. The expression or inhibition of pathway proteins can help understanding effects of a test compound or a test condition on the signaling pathway and find a new drug for medicinal use.

### SUMMARY OF THE INVENTION

The following brief summary is not intended to include all features and aspects of the present invention, nor does it imply that the invention must include all features and aspects discussed in this summary.

Many embodiments of the present invention provide a method of assessing activity of a promoter region. In many other embodiments, a method to assess activity of a promoter region is disclosed comprises culturing a cell comprising a nucleic acid, the nucleic acid comprising a region that encodes a first β-galactosidase fragment operably coupled to a promoter region, under conditions in which the first 3-galactosidase fragment is expressed when the promoter region is active. In further embodiments, the method further comprises contacting the first β-galactosidase fragment, if expressed, with a second β-galactosidase fragment to form an active enzyme complex and detecting the level of the enzymatic activity to assess activity of the promoter region. In another embodiments, the activity of the promoter region may be indicative, and therefore, may be used to assess, the activity of a cellular signaling pathway and/or of endogenous or exogenous (e.g., introduced) transcription factors.

In many embodiments, the present invention provides a method to assess activity of a promoter region comprising culturing a cell comprising a nucleic acid, the nucleic acid comprising a region that encodes an enzyme donor (ED) operably coupled to a promoter region, under conditions in which the ED is expressed when the promoter region is active. In many other embodiments, the method further comprises contacting the ED, if expressed, with an enzyme acceptor (EA) to form a ED-EA complex having enzymatic activity and detecting the level of the enzymatic activity to assess activity of the promoter region. In even further more embodiments, the ED fragment comprises the amino acid sequence set forth in SEQ ID NO: 30, or a variant thereof. In another embodiments, the activity of the promoter region may be indicative, and therefore, may be used to assess, the activity of a cellular signaling pathway and/or of endogenous or exogenous (e.g., introduced) transcription factors.

In further embodiments, a method to assess activity of a promoter region is disclosed wherein the method comprises culturing a cell comprising a nucleic acid, the nucleic acid comprising a region that encodes a first β-galactosidase fragment fused to a carrier protein wherein the first β-galactosidase fragment is operably coupled to a promoter region, under conditions in which the first β-galactosidase fragment-carrier protein fusion is expressed when the promoter region is active. In certain embodiments, the method further comprises contacting the first β-galactosidase fragment, if expressed, with a second β-galactosidase fragment to form an active enzyme complex and detecting the level of the enzymatic activity to assess activity of the promoter region. In many embodiments, the activity of the promoter region may be indicative, and therefore, may be used to assess, the activity of a cellular signaling pathway and/or of endogenous or exogenous (e.g., introduced) transcription factors. In many other embodiments, the carrier protein comprises a domain selected to affect the stability of the ED-carrier protein fusions wherein a domain is selected to increase the stability of the ED-carrier protein fusions as compared to ED-carrier protein fusions lacking the domain or a domain is selected to destabilize the ED-carrier protein fusions as compared to ED-carrier protein fusions lacking the domain. Further, the carrier protein domain targets the ED-carrier protein fusion for proteosomal degradation. The domain comprises a proline (P), glutamic acid (E), serine (S), and threonine (T) (PEST) degradation signal or a CL1 degradation signal.

In further embodiments, a method to assess activity of a promoter region is disclosed, wherein the method comprises culturing a cell comprising a nucleic acid, the nucleic acid comprising a region that encodes an enzyme donor (ED) fragment fused to a carrier protein, wherein the ED fragment is operably coupled to a promoter region, under conditions in which the ED is expressed when the promoter region is active. In certain embodiments, the method further comprises contacting ED, if expressed, with an enzyme acceptor (EA) fragment to form an ED-EA complex with enzymatic activity and detecting the level of the enzymatic activity to assess activity of the promoter region. In many other embodiments, the carrier protein comprises a domain selected to affect the stability of the ED-carrier protein fusions wherein a domain is selected to increase the stability of the ED-carrier protein fusions as compared to ED-carrier protein fusions lacking the domain or a domain is selected to destabilize the ED-carrier protein fusions as compared to ED-carrier protein fusions lacking the domain.

In many embodiments, the carrier protein may possess a detectable enzymatic activity which is not same as the enzymatic activity of β-galactosidase enzymatic activity, wherein the carrier protein enzymatic activity can be detected using known detection methods for that enzymatic activity when the carrier protein is expressed as described in the disclosed methods. In many other embodiments, the carrier protein is expressed under conditions in which the promoter region is active such that enzymatic activity of the carrier protein can be detected using the detection method to assess the activity of the promoter region. In further embodiments, the carrier protein is co-expressed with the expression of ED fragment when the promoter region is active wherein the ED fragment forms a complex with the EA fragment to form an active enzyme complex having enzymatic activity and detecting the level of the enzymatic activity of both the carrier protein and the β-galactosidase enzyme complex to assess activity of the promoter region. In another embodiments, the activity of the promoter region may be indicative, and therefore, may be used to assess, the activity of a cellular signaling pathway and/or of endogenous or exogenous (e.g., introduced) transcription factors.

In various embodiments, a carrier protein may be a natural protein, a mutated protein, a synthetic protein wherein the enzymatic activity of the carrier protein is detected by known detection methods for such a carrier protein. In further embodiments, a carrier protein is mutated such that the mutation renders the enzymatic activity of the carrier protein inactive. In yet further embodiments, the mutated carrier protein may act only as a carrier protein fused with a β-galactosidase enzyme fragment which is operably linked to a promoter region of interest without exhibiting any detectable enzymatic activity when the promoter region of interest is active.

In one embodiment, the present invention provides a method of assessing activity of a promoter region wherein the method comprising culturing a cell comprising a nucleic acid, the nucleic acid comprising a region that encodes a carrier protein fused to ED wherein ED-carrier protein fusion is operably coupled to a promoter region, under conditions in which the ED fragment is expressed along with the expression of the carrier protein when the promoter region is active. In another embodiment, the method further comprises contacting the ED fragment with an EA fragment to form an active enzyme complex having enzymatic activity and detecting the level of the enzymatic activity to assess activity of the promoter region. In yet further embodiment, the method detects the non-β-galactosidase enzymatic activity of the carrier protein and the β-galactosidase enzymatic activity of the ED-EA enzyme complex such that the result is a two point detection method giving assay result from both the carrier protein enzyme activity and the ED-EA fragment complex enzyme activity.

In many embodiment, the present invention provides a method of assessing activity of a promoter region wherein the method comprising culturing a cell comprising a nucleic acid, the nucleic acid comprising a region that encodes a mutated carrier protein fused to an ED fragment wherein the ED-mutated carrier protein fusion is operably coupled to a promoter region, wherein the mutated carrier protein lacks a detectable enzymatic activity. The cell is cultured under conditions in which the ED fragment is expressed when the promoter region is active. In certain embodiments, the method further comprises contacting ED, if expressed, with an EA to form an active enzyme complex having enzymatic activity and detecting the level of enzymatic activity to assess the activity of the promoter region.

In many embodiment, the present invention provides a method of assessing activity of a promoter region wherein the method comprising culturing a cell comprising a nucleic acid, the nucleic acid comprising a region that encodes a carrier protein without any intrinsic enzymatic activity fused to an ED fragment wherein the ED-carrier protein fusion is operably coupled to a promoter region, wherein the carrier protein lacks a detectable enzymatic activity. The cell is cultured under conditions in which the ED fragment is expressed when the promoter region is active. In certain embodiments, the method further comprises contacting ED, if expressed, with an EA to form an active enzyme complex having enzymatic activity and detecting the level of enzymatic activity to assess the activity of the promoter region.

In many embodiments, the present invention provide a method of assessing activity of a promoter region. In further embodiments, a method to assess activity of a promoter region is disclosed comprises culturing a cell comprising a nucleic acid, the nucleic acid comprising a region that encodes a first β-galactosidase fragment operably coupled to a promoter region, introducing a test agent to the culture, wherein the first β-galactosidase fragment is expressed when the promoter region is active. In further embodiments, the method further comprises contacting the first β-galactosidase fragment, if expressed, with a second β-galactosidase fragment to form an active enzyme complex having enzymatic activity and detecting the level of the enzymatic activity to assess activity of the promoter region. In another embodiment, the activity of the promoter region may be indicative, and therefore, may be used to assess, the activity of a cellular signaling pathway and/or of endogenous or exogenous (e.g., introduced) transcription factors.

In many embodiments, the method further comprising contacting the cell with more than one test agent and detecting the effect of test agent by assessing activity of the promoter region in response to the test agent. In many other embodiments, the method further comprises contacting the cell with a first agent and a second agent wherein the cell is first contacted with a first agent affecting the activity of a promoter region of interest, evaluated by detecting the ED-EA complex enzymatic activity; contacting the cell with a second agent, wherein the second agent affects the activity of the first agent, evaluated by detecting an ED-EA complex enzymatic activity and comparing it with the activity of the promoter region when the cell is contacted with first agent only. In various embodiments, the first agent may be an agonist, and the second agent may be an antagonist.

In various embodiments, the cell may be contacted with more than one agent. The more than one agent as disclosed may be introduced into the cell in sequence, such as a first agent is introduced and the a second agent is introduced or in combination such as more than one agent introduced into the cell culture at the same time, to determine the effect of different test agents on the promoter activity of interest. The agent may be a test agent, a small molecule, an agonist, an antagonist, a biologic, an approved drug, an investigational drug, a peptide, a protein, an antibody, a cell, a cell expressing a heterologous protein, a cell expressing an endogenous protein, a product secreted by a cell, a toxin, a natural product, a promoter, an inhibitor or an inverse agonist.

In certain embodiment, the promoter region comprises at least one transcription factor response element (TFRE) for a transcription factor of interest, wherein the activity of the promoter region is indicative of activity of the transcription factor. In certain more embodiments, assessment of the activation level of the transcription factor is based on the detected level of the enzymatic activity of the first β-galactosidase fragment expressed when the promoter region is active.

In certain embodiments, the promoter region comprises a first TFRE and a second TFRE. In certain other embodiments, the promoter region comprises a first TFRE and a second TRFE wherein the first TFRE and the second TFRE are TFREs for the same transcription factor. In even further more embodiments, the promoter region comprises a first TFRE and a second TFRE, wherein the first TFRE and the second TFRE are TFREs for different transcription factors.

In various embodiments, the promoter region comprises at least one TFRE. Further, according to many embodiments, the promoter region comprises two or more than two TFREs. The TFREs included within the promoter region may be the same TFREs such that the TFREs are for the same transcription factor or may be the different TFREs such that different TFREs within the promoter region are for different transcription factor.

In certain embodiment, the promoter region comprises an endogenous promoter region for a gene of interest. In certain more embodiments, assessment of the activation level of the pathway(s) activating the promoter region is based on the detected level of an enzymatic activity of the first β-galactosidase fragment expressed when the promoter region is active.

In further embodiments, the method as disclosed comprise introducing into a cell an expression vector that encodes the transcription factor, and culturing the cell under conditions in which the transcription factor is expressed wherein the promoter region is coupled to the first β-galactosidase fragment. In certain more embodiments, assessment of the activation level of the expression vector encoded transcription factor is based on the detected level of the enzymatic activity of the first β-galactosidase fragment expressed when the promoter region is active.

In other embodiments, the activity of the promoter region is indicative of activity of a cell signaling pathway of interest wherein assessing the activity level of the cell signaling pathway based on the detected level of the enzymatic activity. In many other embodiments, the activity of the transcription factor of interest is indicative of activity of a cell signaling pathway of interest wherein assessing the activity level of the cell signaling pathway based on the detected level of the enzymatic activity.

In many embodiments, the present invention provides a method of assessing activity of a promoter region, comprising contacting a cell with an agent (e.g., a test agent), and assessing the activity level of the promoter region in response to contacting the cell with the agent based on the detected level of the carrier protein enzymatic activity. In more embodiments, the present invention provides a method of assessing activity level of a promoter region, comprising contacting a cell with an agent (e.g., a test agent); assessing the activity level of the promoter region in response to contacting the cell with the agent based on detected level of the enzymatic activity of ED-EA complex and comparing the level of enzymatic activity detected in the presence of the test agent with the level of enzymatic activity of ED-EA complex in the absence of the test agent.

In further embodiments, the present invention provides a method of assessing activity of a promoter region, comprising culturing a cell comprising a nucleic acid, the nucleic acid comprising a region that encodes a first β-galactosidase fragment operably coupled to a promoter region, under conditions in which the first β-galactosidase fragment is expressed when the promoter region is active; contacting the cell with an agent (e.g., a test agent); contacting the first β-galactosidase fragment, if expressed, with a second β-galactosidase fragment to form an active enzyme complex having enzymatic activity and detecting the level of the enzymatic activity to assess activity of the promoter region in response to contacting the cell with the agent and comparing the level of enzymatic activity detected in the presence of the test agent with the level of enzymatic activity in the absence of the test agent or control conditions.

In even further embodiments, the present invention provides a method of assessing activity of a promoter region, comprising culturing a cell comprising a nucleic acid, the nucleic acid comprising a region that encodes an enzyme donor (ED) fragment operably coupled to a promoter region, under conditions in which the ED fragment is expressed when the promoter region is active; contacting the cell with an agent (e.g., a test agent); contacting the ED, if expressed, with EA fragment to form an active enzyme complex having enzymatic activity and detecting the level of the enzymatic activity of ED-EA complex to assess activity of the promoter region in response to contacting the cell with the agent and comparing the level of enzymatic activity detected in the presence of the agent with the level of enzymatic activity in the absence of the agent.

Accordingly, in many embodiments, the method disclosed a nucleic acid encoding a carrier protein fused to the ED, such that ED-carrier protein fusion is expressed when the promoter region is active. In one embodiment, the carrier protein exhibits an enzymatic activity which is not same as the enzymatic activity of the ED-EA complexes. In another embodiment, the carrier protein is a mutated carrier protein exhibiting no detectable enzymatic activity when compared to the wild type carrier protein. In yet another embodiment, the carrier protein lacking any intrinsic enzymatic activity is fused to the ED fragment, such that ED-carrier protein fusion is expressed when the promoter region is active.

In more embodiments, the agent is a small molecule, a protein, a peptide, an antibody, a cell surface protein, a test agent, a cell, a product from a cell (e.g. a product secreted by a cell), an agonist, an inverse agonist, a partial agonist or an antagonist. In many more embodiments, the cell surface protein is present on a cell that does not comprise a nucleic acid comprising a region that encodes the ED fragment of β-galactosidase enzyme. In other embodiments, the cell surface protein is present on a cell that comprise a nucleic acid comprising a region that encodes the ED fragment of R-galactosidase enzyme

In many embodiments, the method further comprises, detecting a level of the enzymatic activity comprising providing a substrate for the ED-EA complexes, wherein a detectable signal is generated upon hydrolysis of the substrate by the ED-EA complexes. In many other embodiments, the detectable signal is a chemiluminescent signal or a biochemiluminescent signal. In further embodiments, the ED and EA are (β-galactosidase fragments wherein the ED fragment comprises a sequence set forth in SEQ ID NO. 30 or a variant thereof that complexes with the EA to form an ED-EA complex having glycosidase hydrolase activity.

In further embodiments, the cell is a mammalian cell, a rodent cell, a human cell, an immune cell, a T cell, a Jurkat cell, a cancer cell, a carcinoma cell, a HepG2 cell, a sarcoma cell or other known cell types.

In various embodiments, the nucleic acid is a plasmid, a chromosome of the cell, a nuclear chromosome, a mitrochondrial chromosome. In other embodiments, the nucleic acid further encodes a carrier protein fused to the ED, such that ED-carrier protein fusions are expressed when the promoter region is active.

In more embodiments, the carrier protein with a detectable enzymatic activity may be a luciferase, a modified luciferase, β-lactamase, alkaline phosphatase, peroxidase, a fluorescent protein or other carrier proteins with a detectable activity.

In many embodiments, the present invention provides an assay to study agonists, antagonists, activators and inhibitors of various pathways and promoter regions.

In even further embodiments, the present invention also discloses cells, compositions, and kits that find use, e.g., in practicing the methods of the present disclosure.

Other features will be apparent from the accompanying figures and from the detailed description that follows.

### BRIEF DESCRIPTION OF THE FIGURES

Example embodiments are illustrated by way of example and no limitation in the tables and in the accompanying figures, like references indicate similar elements and in which:

FIG. 1: Plasmid map of a Nuclear factors of activated T cells (NFAT) Enzyme Fragment Complementation (EFC) reporter construct.

FIG. 2: Expression NFAT EFC reporter construct in U2OS NFAT EFC Reporter cells in response to a cell stimulation cocktail of phorbol 12-myristate 13-acetate (PMA) and ionomycin.

FIG. 3: Plasmid map of NF-kB EFC Reporter construct.

FIG. 4: Response of single-cell clones of U2OS NF-kB EFC reporter cells to cytokine TNFα.

FIG. 5: Screening of inhibitors using U2OS NFkB EFC reporter cell-based assay.

FIG. 6: Testing of TNFα inhibitory activity and potency of two Humira® lots using NFkB EFC Reporter Assay.

FIG. 7: Endogenous CD40 receptor in NFkB EFC reporter assay cells responds robustly to CD40 ligand (CD40L).

FIG. 8: Jurkat NFAT EFC reporter cell respond to OKT3 ligand expressed and presented on the surface of CHO-K1 cells in a co-culture assay.

FIG. 9: Plasmid map for IL2-Promoter-EFC reporter construct.

FIG. 10: Jurkat IL2-promoter EFC reporter cell line detects the stimulation of multiple distinct response elements through activation of different signaling pathways.

FIG. 11: Response of an IL2-promoter EFC reporter construct, comprising a complex, native promoter with multiple different response elements, to intracellular mimics of two different signaling pathways.

FIG. 12: Activity of RORγT transcription factor is decreased by inverse agonist GSK805 in U2OS cells expressing RORγT transcription factor and RORγT EFC reporter plasmid.

FIG. 13: EFC-based NFκB transcriptional reporter assay exhibits better sensitivity to CD40L/CD40 receptor than the Luciferase system.

FIG. 14: EFC-based NFκB transcriptional reporter assay exhibits better sensitivity to TNFα than the Luciferase system.

FIG. 15: Assay results for an NFκB pathway reporter cell line according to one embodiment of the present disclosure. RLU=relative light units.

FIG. 16: Assay results for an NFAT pathway reporter cell line according to one embodiment of the present disclosure. RLU=relative light units.

FIG. 17: Assay results for a STAT3 pathway reporter cell line according to one embodiment of the present disclosure. RLU=relative light units.

FIG. 18: Assay results for NFAT pathway reporter cell line according to one embodiment of the present disclosure. RLU=relative light units.

FIG. 19: Assay results for PD1 pathway reporter assay demonstrating that a pathway reporter assay can be further modified to generate assays for other targets according to one embodiment of the present disclosure. RLU=relative light units.

FIG. 20: Assay results for NFκB pathway reporter assay with a carrier protein coupled promoter according to one embodiment of the present disclosure.

FIG. 21: Assay results for U2OS RANK NF-κB pathway reporter assay according to one embodiment of the present disclosure.

FIG. 22: Assay results for HEK NF-κB pathway reporter assay according to one embodiment of the present disclosure.

FIG. 23: Assay results for HEK CD27-NF-κB pathway reporter assay according to one embodiment of the present disclosure.

FIGS. 24a and 24b: Assay results for U2OS NF-κB reporter cell line and U2OS RANK-NF-κB reporter cell line respectively according to one embodiment of the present disclosure.

Other features of the present embodiments will be apparent from the accompanying figures and from the detailed description that follows.

### DETAILED DESCRIPTION

Provided are methods of assessing activity of a promoter region. The method comprise culturing a cell including a nucleic acid, the nucleic acid, comprising a region that encodes a first β-galactosidase enzyme fragment operably coupled to a promoter region of interest, under conditions in which the first β-galactosidase enzyme fragment is expressed when the promoter region is active wherein the promoter region may become active in response to the cell culture conditions. The method further includes contacting the first β-galactosidase enzyme fragment, if expressed, with a second β-galactosidase enzyme fragment to form an active enzyme complex; detecting the level of enzymatic activity provides an assessment of the activity of the promoter region. Activity of the promoter region may be indicative, and therefore, may be used to assess the activity of a cellular signaling pathway of interest and/or of endogenous or exogenous (e.g., introduced) transcription factors.

Further, the methods include culturing a cell, including a nucleic acid, the nucleic acid including a region that encodes an enzyme donor (ED) operably coupled to a promoter region of interest, under conditions in which the ED is expressed when the promoter region is active. The methods further include contacting the ED, if expressed, with an enzyme acceptor (EA) to form ED-EA complexes having enzymatic activity. The methods further include detecting the level of the enzymatic activity to assess activity of the promoter region. Activity of the promoter region may be indicative, and therefore may be used to assess, the activity of cellular signaling pathways and/or of endogenous or exogenous (e.g., introduced) transcription factors. Also provided are methods that include contacting the cell with an agent (e.g., a test agent), and assessing the activity level of the promoter region in response to contacting the cell with the agent based on the detected level of the enzymatic activity. Cells, compositions, and kits that find use, e.g., in practicing the methods of the present disclosure, are also provided.

The present invention also provides a method of assessing activity of a promoter region in a cell, wherein the cell comprising a nucleic acid, the nucleic acid comprising a carrier protein fused to an enzyme donor (ED) fragment wherein the carrier protein-ED fusion is operably coupled to a promoter region of interest. The method further comprising, culturing the cell under conditions in which the ED is expressed when the promoter region is active; contacting the ED, if expressed, with an enzyme acceptor (EA) to form ED-EA complexes having enzymatic activity, and detecting the level of the enzymatic activity to assess activity of the promoter region of interest wherein the activity of the promoter region may be indicative of activity of a transcription factor of interest and/or a cell signaling pathway of interest.

Before the methods, cells, compositions and kits of the present disclosure are described in greater detail, it is to be understood that the methods, cells, compositions and kits are not limited to particular embodiments described, as such may, of course, vary. It is also to be understood that the terminology used herein is for the purpose of describing particular embodiments only, and is not intended to be limiting, since the scope of the methods, cells, compositions and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, cells, compositions and kits. The upper and lower limits of these smaller ranges may independently be included in the smaller ranges and are also encompassed within the methods, cells, compositions and kits, subject to any specifically excluded limit in the stated range. Where the stated range includes one or both of the limits, ranges excluding either or both of those included limits are also included in the methods, cells, compositions and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, cells, compositions and kits belong. Although any methods, cells, compositions and kits similar or equivalent to those described herein can also be used in the practice or testing of the methods, cells, compositions and kits, representative illustrative methods, cells, compositions and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, cells, compositions and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as an antecedent basis for use of such exclusive terminology as “solely,” “only” and the like in connection with the recitation of claim elements, or use of a “negative” limitation.

It is appreciated that certain features of the methods, cells, compositions and kits, which are, for clarity, described in the context of separate embodiments, may also be provided in combination in a single embodiment. Conversely, various features of the methods, cells, compositions and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, cells, compositions and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

As summarized above, the present disclosure provides methods of assessing activity of a promoter region. The methods include culturing a cell including a nucleic acid, the nucleic acid including a region that encodes an enzyme donor (ED) operably coupled to a promoter region of interest, under conditions in which the ED is expressed when the promoter region is active. The methods further include contacting the ED, if expressed, with an enzyme acceptor (EA) to form ED-EA complexes having enzymatic activity. The methods further include detecting the level of the enzymatic activity to assess activity of the promoter region of interest. The activity of the promoter region may be indicative of activity of a transcription factor of interest and/or a cell signaling pathway of interest. Accordingly, the methods may further include assessing the activity of a transcription factor of interest and/or a cell signaling pathway of interest based on the detected level of the enzymatic activity.

The methods of assessing activity of a promoter region find use in a variety of contexts. For example, the methods find use in determining the activity level of a promoter region when the cell is under a condition of interest. Conditions of interest include, but are not limited to, pH, temperature, a genetic condition of the cell (e.g., one or more mutations (e.g., point mutation, deletion, insertion, and/or the like) in one or more chromosomes of the cell), conditions in which the cell is contacted with an agent (e.g., a test agent), and the like. In some embodiments, the methods of assessing activity of a promoter region include contacting the cell with an agent (e.g., a test agent) during the culturing, and assessing activity of the promoter region (and optionally, transcriptional activity of a gene of interest and/or activity of a cell signaling pathway of interest) in response to contacting the cell with the agent based on the detected level of the enzymatic activity. Such methods find use, e.g., in determining whether the agent effects the activity level of the promoter region (and optionally, transcriptional activity of a gene of interest and/or activity of a cell signaling pathway of interest). The method of assessing activity of a promoter region can also be used to assess an effect of an agonist, an antagonist, a test agent, a transcription factor, an activator of the cell signaling pathway, or an inhibitor of a cell signaling pathway.

Also provided are methods of assessing whether a test agent effects the activity level of a cell signaling pathway of interest. Such methods include culturing a cell in the presence of a test agent, where the cell includes a nucleic acid including a region that encodes an ED operably coupled to a promoter region, under conditions in which the ED is expressed when the promoter region is active, where the activity of the promoter region is indicative of the activity level of the cell signaling pathway of interest. The methods further include contacting the ED, if expressed, with an EA to form ED-EA complexes having enzymatic activity, and detecting the level of the enzymatic activity to assess whether the test agent effects the activity level of the cell signaling pathway of interest.

The methods are based in part on the unexpected finding that the enzyme fragment complementation (EFC)-based reporter assays/systems of the present disclosure exhibit increased sensitivity as compared to existing reporter systems, which rely upon expression of 1) full length (single polypeptide) enzymes such as full-length luciferase, β-galactosidase, chloramphenicol acetyl transferase (CAT); and 2) fluorescent proteins. Additionally, the methods further demonstrates that, For example, as demonstrated in the Experimental section herein below, increased sensitivity for ligand stimulation was observed for the EFC-based reporter assays of the present disclosure as compared to the counterpart luciferase-based assays which rely upon expression of full length (single polypeptide) luciferase enzyme. As such, the assays of the present disclosure constitute an improvement over existing reporter assays with respect to, e.g., the ability of agents (e.g., test agents) to behave more potently in the assays, which in some embodiments results in the assays being more physiologically relevant—that is, more accurately reflecting the effect of the agent (e.g., test agent) on the cell in a natural context, more similar to an in vivo context.

In some embodiments, the sensitivity of the methods is expressed according to the half maximal effective concentration (EC₅₀) value, which in the context of the present disclosure is the concentration of an agent (e.g., a test agent) which induces a response in the cell (as indicated by the level of the enzymatic activity) halfway between the baseline and maximum after exposure of the cell to the agent for a specified exposure time. According to some embodiments, a method of the present disclosure (e.g., a method of assessing the effect of a test agent on a promoter region or cell signaling pathway of interest) exhibits an EC₅₀ value of 100 μg/mL or less, 10 μg/mL or less, 1 μg/mL or less, 100 ng/mL or less, 10 ng/mL or less, 1 ng/mL or less, 100 pg/mL or less, or 10 pg/mL or less. According to some embodiments, a method of the present disclosure (e.g., a method of assessing the effect of a test agent on a promoter region or cell signaling pathway of interest) exhibits an EC₅₀ value of 10 μM or less, 1 μM or less, 100 nM or less, 10 nM or less, 1 nM or less, 100 pM or less, 10 pM or less, or 1 pM or less.

According to some embodiments, the methods of the present disclosure exhibit a potency that is greater as compared to existing reporter systems which rely upon expression of 1) full length (single polypeptide) enzymes such as full-length luciferase, β-galactosidase, chloramphenicol acetyl transferase (CAT); and/or 2) fluorescent proteins. A greater potency is indicated by a smaller value for EC₅₀ as described above. In certain embodiments, the methods of the present disclosure exhibit a potency that is 2:1 or greater, 5:1 or greater, 10:1 or greater, 15:1 or greater, 20:1 or greater, 25:1 or greater, 30:1 or greater, 35:1 or greater, 40:1 or greater, 45:1 or greater, or 50:1 or greater.

As used herein, a “promoter region” is a region of the nucleic acid (e.g., DNA) that includes at least one element (e.g., nucleotide sequence, such as a transcription factor response element (TFRE)) known to regulate transcription. For example, the promoter region may include at least one element known to be bound by a DNA-binding domain of a transcription factor. In certain embodiments, the at least one element is known to regulate expression of one or more genes depending on whether an activated transcription factor is bound to the element. In this way, the combination of the promoter region and the ED-EA reporter system enables interrogation of the activity level of the promoter region, which in turn facilitates identification of conditions that effect expression of the one or more genes known to be regulated by the at least one element in the promoter region. As will be appreciated, activity level of the promoter region may be indicative of the activity level of a transcription factor of interest and/or cell signaling pathway of interest (e.g., transcriptional upregulation and/or downregulation of the one or more genes may be the downstream result of the signaling pathway of interest, e.g., the signaling pathway may regulate the activity of the transcription factor by post-translationally modifying it by phosphorylation, acetylation, ubiquitinylation, and/or other covalent modification), such that the promoter region and the ED-EA reporter system enables interrogation of the activity level of the transcription factor of interest and/or cell signaling pathway of interest, which in turn facilitates identification of conditions that effect the activity level of the transcription factor of interest and/or cell signaling pathway of interest. According to some embodiments, such as conditions include the contacting of the cell with an agent, e.g., a test agent.

Another manner in which a signaling pathway may regulate the activity of a transcription factor is to regulate the concentration of active transcription factor at the site of the promoter associated with ED expression, either through altering its synthesis, its degradation, and/or its subcellular location, all of which may affect the ability of the transcription factor to regulate the promoter region.

Transcription factors of interest include, but are not limited to: an endogenous transcription factor (that is—a transcription factor expressed by the cell from a native/non-introduced nucleic acid of the cell (e.g., a wild-type chromosome of the cell); a heterologous, transfected natural transcription factor (that is—the wild-type form of a transcription factor not otherwise expressed by the cell); a heterologous, transfected recombinant chimeric transcription factor (that is—a transcription factor that includes two or more heterologous domains, e.g., the activation domain of a transcription factor of interest fused to a heterologous DNA binding domain (e.g., GAL4 DNA binding domain) for binding to a generic TFRE (e.g., GAL4/UAS) of the nucleic acid); a heterologous transfected constitutively active transcription factor; or any combination of two or more of such types of transcription factors. According to any such embodiments, the transcription factor may be activated or inactivated by, and may reflect the activity of, an endogenous or engineered cellular signaling pathway.

In certain embodiments, one or more proteins of a cellular pathway or signaling pathway may be genetically altered (e.g. overexpressed, knocked down or knocked out) to allow the pathway to be better studied, to answer specific mechanistic questions or to serve as positive or negative experimental controls. In certain embodiments, genetically altered cellular pathway or signaling pathway may be constitutively active or inactive as needed for an intended experimental purpose.

As summarized above, the methods of the present disclosure may include assessing the activity of a transcription factor of interest and/or cell signaling pathway of interest, where the activity level of the promoter region (and corresponding expression level of the ED) provides a readout for the activity level of the transcription factor of interest and/or cell signaling pathway of interest. In some embodiments, the methods include assessing the activity level of a transcription factor of interest and/or cell signaling pathway of interest in response to contacting the cell with an agent (e.g., a control agent, a test agent, or the like) based on the detected level of the enzymatic activity. By “test agent” is meant an agent (small molecule, peptide, polypeptide, nucleic acid, or the like) which, prior to contacting the cell with the agent, it is unknown whether contacting the cell with the agent will alter the activity level of the transcription factor of interest and/or cell signaling pathway of interest. A test agent may further be meant, but is not limited to, a small molecule, an agonist, an antagonist, a biologic, an approved drug, an investigational drug, a peptide, a protein, an antibody, a cell, a cell expressing a heterologous protein, a cell expressing an endogenous protein, a product secreted by a ell, a toxin, a natural product, a promoter, an inhibitor or an inverse agonist.

A test agent employed according to the methods of the present disclosure may be cell impermeable (e.g., to interrogate whether the test agent alters the activity level of the transcription factor of interest and/or cell signaling pathway of interest via interacting with (e.g., binding) a molecule on the surface of the cell) or cell permeable, e.g., to interrogate whether the test agent alters the activity level of the cell signaling pathway via interacting with (e.g., binding) a molecule on the surface of the cell or a molecule within the cell or a compartment thereof, e.g., a molecule within the cytosol, a molecule on the surface of an organelle, a molecule within an organelle, etc.

As used herein, a “cell signaling pathway” includes a molecule of the cell or series of molecules of the cell (e.g., one or more cell surface molecules and/or one or more intracellular molecules) that respond to an external signal such that the external signal results in the upregulation of expression of one or more genes and/or the downregulation of expression of one or more genes. The upregulation and/or downregulation of the expression of the one or more genes corresponds to the increase and/or decrease in the promoter activity level of the one or more genes. As such, the activity level of a cell signaling pathway may be assessed based on the activity level of a promoter region (or sub-region thereof) which is the downstream target (positive or negative) of signaling through the cell signaling pathway.

As will be appreciated, a particular signaling pathway may be designated/characterized according to a molecule present in the signaling pathway. For example, a signaling pathway may be designated according to a receptor (e.g., cell surface receptor, cytosolic receptor, or the like) that initiates the signaling upon binding to the external signal. Also by way of example, a particular signaling pathway may be designated/characterized according to a molecule “downstream” of a receptor of the external signal and “upstream” of a transcription factor in the signaling pathway. As another example, a particular signaling pathway may be designated/characterized according to a transcription factor (e.g., NFκB, NFAT, STAT3, etc.) in the signaling pathway.

In many embodiments, a cell signaling pathways for which the activity levels may be assessed (e.g., to determine whether a test compound affects the activity level of the signaling pathway) include, but are not limited to, an Akt signaling pathway, an AMP-activated protein kinase (AMPK) signaling pathway, an apoptosis signaling pathway, an epidermal growth factor receptor (EGFR) signaling pathway, an estrogen signaling pathway, a fibroblast growth factor receptor (FGFR) signaling pathway, a growth factor receptor signaling pathway, an insulin signaling pathway, a JAK-STAT signaling pathway, a mitogen-activated protein kinase (MAPK) signaling pathway, a mechanistic target of rapamycin (mTOR) signaling pathway, an NF-κB signaling pathway, a Notch signaling pathway, a nuclear factor of activated T-cells (NFAT) signaling pathway, a p53 signaling pathway, a transforming growth factor β (TGF-β) signaling pathway, a Toll-like receptor (TLR) signaling pathway, a vascular endothelial growth factor (VEGF) signaling pathway, and a Wnt signaling pathway. According to some embodiments, the cell signaling pathway is an NFκB signaling pathway. In certain embodiments, the cell signaling pathway is an STAT (e.g., STAT3 and/or STATS) signaling pathway. According to some embodiments, the cell signaling pathway is an NFAT signaling pathway.

Agents (e.g., test agents) with which the cell may be contacted include, but are not limited to, small molecules, polypeptides (including peptides), nucleic acids, and the like. In some embodiments, the agent is an agonist, an inverse agonist (that is, an agent that binds to the same molecule (e.g., receptor) as an agonist but has the opposite effect of the agonist), a partial agonist (that is, an agent that binds to the same molecule (e.g., receptor) as an agonist and has the same effect as the agonist, but the effect is of lower magnitude), or an antagonist (that is, an agent that binds to the same molecule (e.g., receptor) as an agonist and prevents the binding of the agonist to the molecule, e.g., without affecting the activity of the molecule). By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less. In certain aspects, the small molecule is not made of repeating molecular units such as are present in a polymer.

The terms “polypeptide”, “peptide”, or “protein” are used interchangeably herein to designate a linear series of amino acid residues connected one to the other by peptide bonds between the alpha-amino and carboxy groups of adjacent residues. The amino acids may include the 20 “standard” genetically encodable amino acids, amino acid analogs, or a combination thereof. In some embodiments, when the test agent is a protein, the protein is a soluble protein, e.g., not associated with (bound to or part of) a cell. In other embodiments, the test agent may be an insoluble protein. Examples of insoluble proteins of interest include, but are not limited to, cell surface proteins. As such, in some embodiments, the methods may include exposing the cell to a second cell, and assessing whether a protein on the surface of the second cell—upon contacting of the cell with the cell surface protein—affects the activity level of the cell signaling pathway of interest in the cell. In some embodiments, the cell may be co-cultured with the second cell to contact the cell with the cell surface protein of the second cell. According to some embodiments, the cell surface protein of the second cell (e.g. the cell-surface ligand) may be isolated and purified from the second cell and contacted with the cell (the responding cell) either as a soluble, soluble and cross-linked, or when coated onto a solid support, e.g., the surface of beads or a tissue culture plate.

In some embodiments, when the agent is a nucleic acid, the agent is an oligonucleotide. As used herein, an “oligonucleotide” is a single-stranded multimer of nucleotides from 2 to 500 nucleotides, e.g., 2 to 200 nucleotides. Oligonucleotides may be synthetic or may be made enzymatically, and, in some embodiments, are 5 to 50 nucleotides in length (e.g., 9 to 50 nucleotides in length). Oligonucleotides may contain ribonucleotide monomers (i.e., may be oligoribonucleotides or “RNA oligonucleotides”) or deoxyribonucleotide monomers (i.e., may be oligodeoxyribonucleotides or “DNA oligonucleotides”). Oligonucleotides may be 5 to 9, 10 to 20, 21 to 30, 31 to 40, 41 to 50, 51 to 60, 61 to 70, 71 to 80, 80 to 100, 100 to 150 or 150 to 200, up to 500 or more nucleotides in length, for example. In some embodiments, when the agent is a nucleic acid, the agent is a short interfering RNA (siRNA), a microRNA (miRNA), a morpholino, and/or the like. Approaches for designing and delivering siRNAs, miRNAs, morpholinos, etc. for targeting a particular mRNA are known and described, e.g., in Monsoori et al. (2014) Adv Pharm Bull. 4(4):313-321; Xin et al. (2017) Mol Cancer 16:134; Chakraborty et al. (2017) Mol Ther Nucleic Acids 8:132-143; and Ahmadzada et al. (2018) Biophys Rev. 10(1):69-86.siRNAs, miRNAs, morpholinos, etc. may be designed based on the known sequence of an mRNA to be targeted and using available tools, e.g., siRNA Wizard from Invivogen, siDESIGN Center from Dharmacon, BLOCK-iT™ RNAi Designer from Invitrogen, miR-Synth available at microrna.osumc.edu/mir-synth, WMD3—Web MicroRNA Designer, a morpholino design tool provided by Gene Tools, etc.

In some embodiments, the cell is contacted with an agent (e.g., a test agent), where the agent is part of a library of agents, e.g., a small molecule library, polypeptide library, siRNA library, or the like. Such methods may further include performing the method in high throughput, where cells are provided to wells of a tissue culture plate (e.g., 4-, 6-, 8-, 12-, 24-, 48-, 96-, 384-, 1536-well tissue culture plate, or the like), the cells are contacted with one or a subset of test agents from a library of test agents (e.g., a small molecule library, polypeptide library, siRNA library, etc.), and the methods include identifying agents that affect the activity level of a signaling pathway of interest based on the detected enzymatic activity level.

According to some embodiments, the promoter region includes a transcription factor response element (TFRE) for a transcription factor of interest, and the activity of the promoter region is indicative of activity of the transcription factor. In some embodiments, the methods include assessing the activation level of the transcription factor based on the detected level of the enzymatic activity. In certain embodiments, a naturally-occurring transcription factor of interest may be expressed or overexpressed in the cells (e.g. if absent or present at lower levels than ideal for the assay). In certain embodiments, the transcription factor is a generic transcription factor, a mutated transcription factor to be constitutively active or inactive. In certain embodiments, a transcription factor may be knocked down or knocked out. In certain embodiments, the transcription factor is a chimeric transcription factor that includes the activation domain of the transcription factor of interest fused to a heterologous nucleic acid binding domain that binds to the TFRE. The TFRE may be a TFRE to which the DNA binding domain of a wild-type transcription factor of interest binds (e.g., a TFRE to which wild-type STAT3 binds in a method that includes assessing the activity level of STAT3). In some embodiments, the TFRE is a “generic” TFRE, meaning that the TFRE is one that may be employed in a reporter assay system for assessing the activity level of various transcription factors that, in nature, bind to different TFREs. For example, in some embodiments, the cell expresses a chimeric transcription factor that includes the activation domain of a transcription factor of interest fused to a heterologous nucleic acid binding domain that binds to the generic TFRE. In this way, the same nucleic acid may be used in EFC reporter assays for assessing the activity levels of transcription factors that, in nature, do not bind to the same TFREs. A non-limiting example of a generic TFRE that may be employed is a GAL4/upstream activating sequence (GAL4/UAS), where the activation domain of a transcription factor of interest (e.g., NFκB, STAT3, NFAT, ELK1, etc.) is fused to a GAL4 DNA binding domain, enabling the assessment of activation of the transcription factor of interest without requiring the native TFRE for the transcription factor of interest. In some embodiments, the methods include introducing into the cell an expression vector that encodes the transcription factor, and culturing the cell under conditions in which the transcription factor is expressed.

In certain embodiments, the promoter region includes a single transcription factor response element (TFRE). The single TFRE may be a TFRE that binds to and responds to activation of a single transcription factor (e.g., class A, an example of which is an NF-kB response element) as exemplified in certain examples in the Experimental section below. Such embodiments find use, e.g., in isolating a particular TFRE of interest to determine conditions that effect the activity of that TFRE in isolation (that is—without interference from other TFREs), and/or determining conditions that affect activation or inactivation of a transcription factor to which the TFRE binds and responds. In certain other embodiments, the promoter region includes more than one transcription factor response element (TFRE)

According to some embodiments, the promoter region comprises at least one TFRE. Further, according to many embodiments, the promoter region comprises two or more than two TFREs. The TFREs included within the promoter region may be the same TFREs such that the TFREs are for the same transcription factor or may be the different TFREs such that different TFREs within the promoter region are for different transcription factor. The TFREs may be introduced into the cell in combination or sequentially. In certain embodiments, the promoter region includes a first TFRE and a second TFRE, where the first and second TFREs are different, e.g., TFREs that bind to and respond to the activation and/or deactivation of different transcription factors. Promoter regions that include two or more TFREs find use, e.g., when it is desirable for the promoter region to mimic a naturally-occurring, wild-type promoter region having multiple TFREs that bind to and respond to the activation and/or deactivation of different transcription factors (e.g., class B, an example of which is the IL-2 gene promoter having at least six different transcription factor-specific response elements) as exemplified in certain examples in the Experimental section below. In some embodiments, the promoter region mimics a subset of all TFREs present in a naturally-occurring, wild-type promoter region having multiple TFREs that bind to and respond to the activation and/or deactivation of different transcription factors. In some embodiments the promoter region includes a TFRE which is an enhancer. By “enhancer” is meant a cis-acting DNA sequence that can be bound by one or more proteins to increase gene transcription, and which may be located up to 1 Mb away from the region encoding the ED. The ability to use different specific promoter regions of either class A or class B allows broad application of the present methods to different biological questions, as well as screening for conditions that produce a desired result, e.g., activation or inactivation of a transcription factor of interest, activation or inactivation of expression of a gene of interest, activation or inactivation of a cell signaling pathway of interest, and/or the like.

As summarized above, the methods include culturing a cell under conditions in which the ED is expressed when the promoter region is active. By “active” is meant the promoter region is in a state that permits a detectably elevated level of ED expression above background. Such a state may be an “unbound” state in which a detectably elevated level of ED expression above background occurs when no transcription factors are bound to the promoter region. Such a state may also be a state in which a detectably elevated level of ED expression above background occurs when one or more transcription factors are bound to the promoter region (e.g., upon activation of one or more of the transcription factors themselves), and where binding of the one or more transcription factors is required for the detectably elevated level of ED expression, and/or up regulates (induces) or down regulates the level of ED expression compared to the level of ED expression when the promoter region is in the unbound state.

The conditions for culturing the cell such that the ED is expressed when the promoter region is active may vary. Such conditions may include culturing the cell in a suitable container (e.g., a cell culture plate or well thereof), in suitable medium (e.g., cell culture medium, such as DMEM, RPMI, MEM, IMDM, DMEM/F-12, or the like) at a suitable temperature (e.g., 32° C.-42° C., such as 37° C.) and pH (e.g., pH 7.0-7.7, such as pH 7.4) in an environment having a suitable percentage of CO₂, e.g., 3% to 10%, such as 5%). Non-limiting examples of cell culture conditions that may be employed are described in the Experimental section below.

The cell employed in the methods may be any suitable cell. In certain embodiments, the type of cell is selected based on a biological process of interest. By way of example, if one wishes to practice the present methods to investigate conditions that effect T cell activation, the cell may be an activatable T cell, e.g., a Jurkat cell. According to some embodiments, the cell is a type of cell employed by those skilled in the art to interrogate a cell signaling pathway of interest. In certain embodiments, the cell is a type of cell employed by those skilled in the art to interrogate a cell containing certain specific cellular and molecular components of interest such as a certain receptor or downstream signaling molecule in a pathway of interest, e.g., protein kinase, adapter, transcription factor, etc.

According to some embodiments, the cell is a primary cell. By “primary cell” is meant a cell obtained directly from living tissue (e.g., biopsy material) and established for growth in vitro. In some embodiments, the cell is from a cell line. Non-limiting examples of such cell lines include Jurkat, U2OS, HepG2, HeLa, MCF-7, PC-12, PBMC, HUVECs, HEK-293, COS-7, BHK-21, HEp-2, HT-1080, MDCK, and the like. According to some embodiments, the cell is an epithelial cell, a mesothelial cell, or an endothelial cell. In some embodiments, the cell is an immune cell. Non-limiting examples of immune cells that may be employed include T cells, B cells, natural killer (NK) cells, macrophages, monocytes, neutrophils, dendritic cells, mast cells, basophils, and eosinophils. In certain embodiments, the immune cell is a T cell. Examples of T cells include naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells (T_{H}, T_{H}1, T_{H}2, T_{H}17), CD4+ T cells, CD8+ T cells, virus-specific T cells, alpha beta T cells (T_{αβ}), and gamma delta T cells (T_{γδ}).

According to some embodiments, the cell is a cancer cell. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a primary tumor, a metastatic tumor, a cancer cell line, and the like. In certain aspects, the cancer cell is a carcinoma cell. Carcinoma cells of interest include, but are not limited to, HepG2 cells. In certain aspects, the cancer cell is a sarcoma cell. Non-limiting examples of sarcoma cells include osteosarcoma cells, such as U2OS cells.

The nucleic acid employed in the present methods may be any nucleic acid suitable for operably coupling the promoter region to the region that encodes the ED. In some embodiments, the nucleic acid is stably integrated into the chromosomal DNA of the cell, e.g., non-specifically or site-specifically. In some embodiments, the nucleic acid is an episome (or “episomal”). By “episome” or “episomal” is meant a nucleic acid (e.g., DNA) molecule that replicates independently of the cell's chromosomal DNA. A non-limiting example of an episome that may be employed in the present methods is a plasmid. When the nucleic acid is an episome (e.g., a plasmid), the episome may include one or more elements in addition to the promoter region and the region that encodes the ED. For example, a plasmid may include an origin of replication, one or more regions that encode a protein that confers antibiotic resistance to the cell (e.g., ampicillin resistance (AmpR), hygromycin resistance, and/or the like), one or more poly(A) signals, a pause site, an SV40 late poly(A) signal, an SV40 enhancer, an SV40 early promoter, etc., and any desired combination of such elements. A plasmid introducing the nucleic acid for episomal or chromosomally-integrated expression may be adjacent and genetically linked to an antibiotic-selectable marker which can be used to select only for cells which are stably expressing the nucleic acid. A plasmid introducing the nucleic acid may be delivered by a viral vector or may be transfected with chemical reagents, by electroporation, or any other suitable approach.

Nucleotide sequences of plasmids (including plasmids employed in the Experimental section below) and elements/subsequences thereof that find use in practicing the methods of the present disclosure are provided in Table 1 below:

TABLE 1
Nucleotide Sequences

| | |
|---|---|
| ePL β-galactosidase | AATTCACTGGCCGTCGTTTTACAACGTCGTGACTGGGAAAAC |
| ED (SEQ ID NO: 1) | CCTGGCGTTACCCAACTTAATCGCCTTGCAGCACATCCCCCTT |
| | TCGCCAGCTGGCGTAATAGCGAAGAGGCCCGCACCGATCGC |
| Minimal CMV | AGAGGGTATATAATGGAAGCTCGACTTCCAG |
| promoter (SEQ ID | |
| NO: 2) | |
| PEST protein | AATTCTCACGGCTTCCCTCCCGAGGTGGAGGAGCAGGCCGCC |
| destabilizing | GGCACCCTGCCCATGAGCTGCGCCCAGGAGAGCGGCATGGA |
| sequence (SEQ ID | TAGACACCCTGCTGCTTGCGCCAGCGCCAGGATCAACGTC |
| NO: 3) | |
| hCL1 protein | GCTTGCAAGAACTGGTTCAGTAGCTTAAGCCACTTTGTGATC |
| destabilizing | CACCTT |
| sequence (SEQ ID | |
| NO: 4) | |
| NFAT response | GGAGGAAAAACTGTTTCATACAGAAGGCGT |
| element (SEQ ID | |
| NO: 5) | |
| NFkB response | GGGAATTTCCGGGGACTTTCCGGGAATTTCCGGGGACTTTCC |
| element (SEQ ID | GGGAATTTCC |
| NO: 6) | |
| RORgT response | GGTAAGTAGGTCAT |
| element (SEQ ID | |
| NO: 7) | |
| Interferon (IFR1 | AGCCTGATTTCCCCGAAATGACGGC |
| GAS) Response | |
| Element (SEQ ID | |
| NO: 8) | |
| STAT3 response | CATTTCCCGTAAATCGTCG |
| element (SEQ ID | |
| NO: 9) | |
| STAT5 response | AGTTCTGAGAAAAGT |
| element (SEQ ID | |
| NO: 10) | |
| IL-2 promoter SEQ | CTTTTCTGAGTTACTTTTGTATCCCCACCCCCTTAAAGAAAGG |
| IDNO: 11) | AGGAAAAACTGTTTCATACAGAAGGCGTTAATTGCATGAATT |
| | AGAGCTATCACCTAAGTGTGGGCTAATGTAACAAAGAGGGA |
| | TTTCACCTACATCCATTCAGTCAGTCTTTGGGGGTTTAAAGAA |
| | ATTCCAAAGAGTCATCAGAAGAGGAAAAATGAAGGTAATGT |
| | TTTTTCAGACAGGTAAAGTCTTTGAAAATATGTGTAATATGT |
| | AAAACATTTTGACACCCCCATAATATTTTTCCAGAATTAACA |
| | GTATAAATTGCATCTCTTGTTCAAGAGTTCCCTATCACTCTCT |
| | TTAATCACTACTCACAGTAACCTCAACTCCTGCCA |
| IL2 promoter DNA | ACCCCCTTAAAGAAAGGAGGAA |
| response element | |
| (SEQ ID NO: 12) | |
| (NFAT & AP1) | |
| IL2 promoter DNA | GGAGGAAAAACTGTTTCATACAGAAGGCGT |
| response element | |
| (SEQ ID NO: 13) | |
| (NFAT) | |
| IL2 promoter DNA | AATTGCATGAA |
| response element | |
| (SEQ ID NO: 14) | |
| (OCT) | |
| IL2 promoter DNA | GGGATTTCACC |
| response element | |
| (SEQ ID NO: 15) | |
| (NFκB) | |
| IL2 promoter DNA | ATGAAGGTAATGTTTTTTCAG |
| response element | |
| (SEQ ID NO: 16) | |
| (NFAT & AP1) | |
| IL2 promoter DNA | GTCTTTGAAAATATGTGTAAT |
| response element | |
| (SEQ ID NO: 17) | |
| (NFAT & AP1) | |
| IL2 promoter DNA | AAACATTTTG |
| response element | |
| (SEQ ID NO: 18) | |
| (OCT) | |
| IL2 promoter DNA | TAATATTTTT |
| response element | |
| (SEQ ID NO: 19) | |
| (NFAT) | |
| IL-2 core promoter | CAGAATTAACAGTATAAATTGCATCTCTTGTTCAAGAGTTCC |
| (SEQ ID NO: 20) | CTATCACTCT |
| (TATA box | |
| underlined) | |
| SEQ ID NO. 21 | actcgtccttatcaatattattgaagcatttatcagggttactagtacgtctctcaaggataagtaagtaat |
| NFAT Reporter | attaaggtacgggaggtattggacaggccgcaataaaatatctttattttcattacatctgtgtgttggtttt |
| Plasmid used is | ttgtgtgaatcgatagtactaacatacgctctccatcaaaacaaaacgaaacaaaacaaactagcaaaa |
| SEQ ID NO: 21- | taggctgtccccagtgcaagtgcaggtgccagaacatttctctggcctaacTGGCCGCTTACC |
| XXX-SEQ ID NO. | TGAGCTCGCTAGCGGAGGAAAAACTGTTTCATACAGAAGGC |
| 33 | GTGGAGGAAAAACTGTTTCATACAGAAGGCGTGGAGGAAAA |
| (“XXX” represents | ACTGTTTCATACAGAAGGCGTAGATCTACTAGAGGGTATATA |
| nucleotide sequence | ATGGAAGCTCGACTTCCAGCTTGGCAATCCGGTACTGTTGGT |
| encoding carrier | AAAGCCACC |
| protein of interest) | -XXX- |
| SEQ ID NO. 33 | AAATTCTCACGGCTTCCCTCCCGAGGTGGAGGAGCAGGCCGC |
| | CGGCACCCTGCCCATGAGCTGCGCCCAGGAGAGCgGCATGG |
| | ATAGACACCCTGCTGCTTGCGCCAGCGCCAGGATCAACGTCT |
| | TCGAATTGGGAGGTGGCGGTAGCGGAGGTGGCGGTAGCCTC |
| | GAGAGCTCCAATTCACTGGCCGTCGTTTTACAACGTCGTGAC |
| | TGGGAAAACCCTGGCGTTACCCAACTTAATCGCCTTGCAGCA |
| | CATCCCCCTTTCGCCAGCTGGCGTAATAGCGAAGAGGCCCGC |
| | ACCGATCGCTAGTGAGGCCGGccgcttcgagcagacatgataagatacattgatg |
| | agtttggacaaaccacaactagaatgcagtgaaaaaaatgctttatttgtgaaatttgtgatgctattgctt |
| | tatttgtaaccattataagctgcaataaacaagttaacaacaacaattgcattcattttatgtttcaggttca |
| | gggggaggtgtgggaggttttttaaagcaagtaaaacctctacaaatgtggtaaaatcgataaggatcc |
| | gtttgcgtattgggcgctcttccgctgatctgcgcagcaccatggcctgaaataacctctgaaagagga |
| | acttggttagctaccttctgaggcggaaagaaccagctgtggaatgtgtgtcagttagggtgtggaaa |
| | gtccccaggctccccagcaggcagaagtatgcaaagcatgcatctcaattagtcagcaaccaggtgt |
| | ggaaagtccccaggctccccagcaggcagaagtatgcaaagcatgcatctcaattagtcagcaacca |
| | tagtcccgcccctaactccgcccatcccgcccctaactccgcccagttccgcccattctccgccccatg |
| | gctgactaattttttttatttatgcagaggccgaggccgcctctgcctctgagctattccagaagtagtga |
| | ggaggcttttttggaggcctaggcttttgcaaaaagctcgattcttctgacactagcgccaccatgaaga |
| | agcccgaactcaccgctaccagcgttgaaaaatttctcatcgagaagttcgacagtgtgagegaccts |
| | atgcagttgtcggagggcgaagagagccgagccttcagcttcgatgtcggcggacgcggctatgta |
| | ctgcgggtgaatagctgcgctgatggcttctacaaagaccgctacgtgtaccgccacttcgccagcgc |
| | tgcactacccatccccgaagtgttggacatcggcgagttcagcgagagcctgacatactgcatcagta |
| | gacgcgcccaaggcgttactctccaagacctccccgaaacagagctgcctgctgtgttacagcctgtc |
| | gccgaagctatggatgctattgccgccgccgacctcagtcaaaccagcggcttcggcccattcgggc |
| | cccaaggcatcggccagtacacaacctggcgggatttcatttgcgccattgctgatccccatgtctacc |
| | actggcagaccgtgatggacgacaccgtgtccgccagcgtagctcaagccctggacgaactgatgc |
| | tgtgggccgaagactgtcccgaggtgcgccacctcgtccatgccgacttcggcagcaacaacgtcct |
| | gaccgacaacggccgcatcaccgccgtaatcgactggtccgaagctatgttcggggacagtcagtac |
| | gaggtggccaacatcttcttctggcggccctggctggcttgcatggagcagcagactcgctacttcga |
| | gcgccggcatcccgagctggccggcagccctcgtctgcgagcctacatgctgcgcatcggcctgga |
| | tcagctctaccagagcctcgtggacggcaacttcgacgatgctgcctsggctcaaggccgctgcgat |
| | gccatcgtccgcagcggggccggcaccgtcggtcgcacacaaatcgctcgccggagcgcagccgt |
| | atggaccgacggctgcgtcgaggtgctggccgacagcggcaaccgccggcccagtacacgaccg |
| | cgcgctaaggaggtaggtcgagtttaaactctagaaccggtcatggccgcaataaaatatctttattttc |
| | attacatctgtgtgttggttttttgtgtgttcgaactagatgctgtcgaccgatgcccttgagagccttcaac |
| | ccagtcagctccttccggtgggcgcggggcatgactatcgtcgccgcacttatgactgtcttctttatca |
| | tgcaactcgtaggacaggtgccggcagcgctcttccgcttcctcgctcactgactcgctgcgctcggt |
| | cgttcggctgcggcgagcggtatcagctcactcaaaggcggtaatacggttatccacagaatcaggg |
| | gataacgcaggaaagaacatgtgagcaaaaggccagcaaaaggccaggaaccgtaaaaaggccg |
| | cgttgctggcgtttttccataggctccgcccccctgacgagcatcacaaaaatcgacgctcaagtcaga |
| | ggtggcgaaacccgacaggactataaagataccaggcgtttccccctggaagctccctcgtgcgctc |
| | tcctgttccgacccthccgcttaccggatacctgtccgcctttctcccttcgggaagcgtggcgctttctc |
| | atagctcacgctgtaggtatctcagttcggtgtaggtcgttcgctccaagctgggctgtgtgcacgaac |
| | cccccgttcagcccgaccgctgcgccttatccggtaactatcgtcttgagtccaacccggtaagacac |
| | gacttatcgccactggcagcagccactggtaacaggattagcagagcgaggtatgtaggcggtgcta |
| | cagagttatgaagtggtggcctaactacggctacactagaagaacagtatttggtatctgcgactgct |
| | gaagccagttaccttcggaaaaagagttggtagctcttgatccggcaaacaaaccaccgctggtagc |
| | ggtggtttttttgtttgcaagcagcagattacgcgcagaaaaaaaggatctcaagaagatcctttgatctt |
| | ttctacggggtctgacgctcagtggaacgaaaactcacgttaagggattttggtcatgagattatcaaaa |
| | aggatcttcacctagatccttttaaattaaaaatgaagttttaaatcaatctaaagtatatatgagtaaactt |
| | ggtctgacagcggccgcaaatgctaaaccactgcagtggttaccagtgcttgatcagtgaggcaccg |
| | atctcagcgatctgcctatttcgttcgtccatagtggcctgactccccgtcgtgtagatcactacgattcgt |
| | gagggcttaccatcaggccccagcgcagcaatgatgccgcgagagccgcgttcaccggcccccga |
| | tttgtcagcaatgaaccagccagcagggagggccgagcgaagaagtggtcctgctactttgtccgcc |
| | tccatccagtctatgagctgctgtcgtgatgctagagtaagaagttcgccagtgagtagtttccgaaga |
| | gttgtggccattgctactggcatcgtggtatcacgctcgtcgttcggtatggcttcgttcaactctggttcc |
| | cagcggtcaagccgggtcacatgatcacccatattatgaagaaatgcagtcagctccttagggcctcc |
| | gatcgttgtcagaagtaagttggccgcggtgttgtcgctcatggtaatggcagcactacacaattctctt |
| | accgtcatgccatccgtaagatgcttttccgtgaccggcgagtactcaaccaagtcgttttgtgagtagt |
| | gtatacggcgaccaagctgctcttgcccggcgtctatacgggacaacaccgcgccacatagcagtac |
| | tttgaaagtgacatcatcgggaatcgttcttcggggcggaaagactcaaggatcttgccgctattgag |
| | atccagttcgatatagcccactcttgcacccagttgatcttcagcatcttttactttcaccagcgtttcggg |
| | gtgtgcaaaaacaggcaagcaaaatgccgcaaagaagggaatgagtgcgacacgaaaatgttggat |
| | gctcat |
| SEQ ID NO. 22 | actcgtcctttttcaatattattgaagcatttatcagggttactagtacgtctctcaaggataagtaagtaat |
| IL-2 Reporter | attaaggtacgggaggtattggacaggccgcaataaaatatctttattttcattacatctgtgtttggtttt |
| Plasmid is SEQ ID | ttgtgtgaatcgatagtactaacatacgctctccatcaaaacaaaacgaaacaaaacaaactagcaaaa |
| NO: 22-XXX-SEQ | taggctgtccccagtgcaagtgcaggtgccagaacatttctctggcctaactggccggtacCTTTT |
| ID NO, 34 | CTGAGTTACTTTTGTATCCCCACCCCCTTAAAGAAAGGAGGA |
| (“XXX” represents | AAAACTGTTTCATACAGAAGGCGTTAATTGCATGAATTAGAG |
| nucleotide sequence | CTATCACCTAAGTGTGGGCTAATGTAACAAAGAGGGATTTCA |
| encoding carrier | CCTACATCCATTCAGTCAGTCTTTGGGGGTTTAAAGAAATTCC |
| protein of interest) | AAAGAGTCATCAGAAGAGGAAAAATGAAGGTAATGTTTTTTC |
| | AGACAGGTAAAGTCTTTGAAAATATGTGTAATATGTAAAACA |
| | TTTTGACACCCCCATAATATTTTTCCAGAATTAACAGTATAAA |
| | TTGCATCTCTTGTTCAAGAGTTCCCTATCACTCTCTTTAATCA |
| | CTACTCACAGTAACCTCAACTCCTGCCAgctag |
| | -XXX- |
| SEQ ID NO. 34 | aagcttGGAGGTGGCGGTAGCGGAGGTGGCGGTAGCCTCGAGAG |
| | CTCCAATTCACTGGCCGTCGTTTTACAACGTCGTGACTGGGA |
| | AAACCCTGGCGTTACCCAACTTAATCGCCTTGCAGCACATCC |
| | CCCTTTCGCCAGCTGGCGTAATAGCGAAGAGGCCCGCACCGA |
| | TCGCTAGggccggCCgcttcgagcagacatgataagatacattgatgagtttggacaaacca |
| | caactagaatgcagtgaaaaaaatgctttatttgtgaaatttgtgatgctattgctttatttgtaaccattata |
| | agctgcaataaacaagttaacaacaacaattgcattcattttatgtttcaggttcagggggaggtgtggg |
| | aggttttttaaagcaagtaaaacctctacaaatgtggtaaaatcgataaggatccgtttgcgtattgggcg |
| | ctcttccgctgatctgcgcagcaccatggcctgaaataacctctgaaagaggaacttggttagctacctt |
| | ctgaggcggaaagaaccagctgtggaatgtgtgtcagttagggtgtggaaagtccccaggctcccca |
| | gcaggcagaagtagcaaagcatgcatctcaattagtcagcaaccaggtgtggaaagtccccaggct |
| | ccccagcaggcagaagtatgcaaagcatgcatctcaattagtcagcaaccatagtcccgcccctaact |
| | ccgcccatcccgcccctaactccgcccagttccgcccattctccgccccatggctgactaattttttttatt |
| | tatgcagaggccgaggccgcctctcctctgagctattccagaagtagtgaggaggcttttttggagg |
| | cctaggcttttgcaaaaagctcgattcttctgacactagcgccaccatgaagaagcccgaactcaccg |
| | ctaccagcgttgaaaaatttctcatcgagaagttcgacagtgtgagcgacctgatgcagttgtcggagg |
| | gcgaagagagccgagccttcagcttcgatgtcggcggacgcggctatgtactgcgggtgaatagct |
| | gcgctgatggcttctacaaagaccgctacgtgtaccgccacttcgccagcgctgcactacccatcccc |
| | gaagtgttggacatcggcgagttcagcgagagcctgacatactgcatcagtagacgcgcccaaggc |
| | gttactctccaagacctccccgaaacagagctgcctgctgtgttacagcctgtcgccgaagctatggat |
| | gctattgccgccgccgacctcagtcaaaccagcggcttcggcccattcgggccccaaggcatcggc |
| | cagtacacaacctggcgggatttcatttgcgccattgctgatccccatgtctaccactggcagaccgtg |
| | atggacgacaccgtgtccgccagcgtagctcaagccctggacgaactgatgctgtgggccgaagac |
| | tgtcccgaggtgcgccacctcgtccatgccgacttcggcagcaacaacgtcctgaccgacaacggc |
| | cgcatcaccgccgtaatcgactggtccgaagctatgttcggggacagtcagtacgaggtggccaaca |
| | tcttcttctggcggccctggctggcttgcatggagcagcagactcgctacttcgagegccggcatccc |
| | gagctggccggcagccctcgtctgcgagcctacatgctgcgcatcggcctggatcagctctaccaga |
| | gcctcgtggacggcaacttcgacgatgctgcctgggctcaaggccgctgcgatgccatcgtccgcag |
| | cggggccggcaccgtcggtcgcacacaaatcgctcgccggagcgcagccgtatggaccgacggct |
| | gcgtcgaggtgctggccgacagcggcaaccgccggcccagtacacgaccgcgcgctaaggaggt |
| | aggtcgagtttaaactctagaaccggtcatggccgcaataaaatatctttattttcattacatctgtgtgttg |
| | gttttttgtgtgttcgaactagatgctgtcgaccgatgcccttgagagccttcaacccagtcagctccttc |
| | cggtgggcgcggggcatgactatcgtcgccgcacttatgactgtcttctttatcatgcaactcgtagga |
| | caggtgccggcagcgctcttccgcttcctcgctcactgactcgctgcgctcggtcgttcggctgcggc |
| | gagcggtatcagctcactcaaaggcggtaatacggttatccacagaatcaggggataacgcaggaaa |
| | gaacatgtgagcaaaaggccagcaaaaggccaggaaccgtaaaaaggccgcgttgctggcgttttt |
| | ccataggaccgcccccagacgagcatcacaaaaatcgacgctcaagtcagaggtggcgaaaccc |
| | gacaggactataaagataccaggcgtttccccctggaagctccctcgtgcgctctcctgttccgaccct |
| | gccgcttaccggatacctgtccgcctttctcccttcgggaagcgtggcgctttctcatagctcacgctgt |
| | aggtatctcagttcggtgtaggtcgttcgctccaagctgggctgtgtgcacgaaccccccgttcagccc |
| | gaccgctgcgccttatccggtaactatcgtcttgagtccaacccggtaagacacgacttatcgccactg |
| | gcagcagccactggtaacaggattagcagagcgaggtatgtaggcggtgctacagagttcttgaagt |
| | ggtggcctaactacggctacactagaagaacagtatttggtatctgcgctctgctgaagccagttacctt |
| | cggaaaaagagttggtagctcttgatccggcaaacaaaccaccgctggtagcggtggtttttttgtttgc |
| | aagcagcagattacgcgcagaaaaaaaggatctcaagaagatcctttgatcttttctacggggtctgac |
| | gctcagtggaacgaaaactcacgttaagggattttggtcatgagattatcaaaaaggatcttcacctaga |
| | tccttttaaattaaaaatgaagttttaaatcaatctaaagtatatatgagtaaacttggtctgacagcggcc |
| | gcaaatgctaaaccactgcagtggttaccagtgcttgatcagtgaggcaccgatctcagcgatctgcct |
| | atttcgttcgtccatagtggcctgactccccgtcgtgtagatcactacgattcgtgagggcttaccatcag |
| | gccccagcgcagcaatgatgccgcgagagccgcgttcaccggcccccgatttgtcagcaatgaacc |
| | agccagcagggagggccgagegaagaagtggtcctgctactttgtccgcctccatccagtctatgag |
| | ctgctgtcgtgatgctagagtaagaagttcgccagtgagtagtttccgaagagttgtggccattgctact |
| | ggcatcgtggtatcacgctcgtcgttcggtatggcttcgttcaactaggttcccagcggtcaagccgg |
| | gtcacatgatcacccatattatgaagaaatgcagtcagctccttagggcctccgatcgttgtcagaagta |
| | agttggccgcggtgttgtcgctcatggtaatggcagcactacacaattctcttaccgtcatgccatccgt |
| | aagatgcttttccgtgaccggcgagtactcaaccaagtcgttttgtgagtagtgtatacggcgaccaag |
| | ctgctcttgcccggcgtctatacgggacaacaccgcgccacatagcagtactttgaaagtgctcatcat |
| | cgggaatcgttcttcggggcggaaagactcaaggatcttgccgctattgagatccagttcgatatagcc |
| | cactcttgcacccagttgatcttcagcatcttttactttcaccagcgtttcggggtgtgcaaaaacaggca |
| | agcaaaatgccgcaaagaagggaatgagtgcgacacgaaaatgttggatgctcat |
| SEQ ID NO: 23 | actcgtcctttttcaatattattgaagcatttatcagggtttactagtacgtctctcaaggataagtaagtaat |
| Interferon-gamma | attaaggtacgggaggtattggacaggccgcaataaaatatctttattttcattacatctgtgtgttggtttt |
| Reporter Plasmid is | ttgtgtgaatcgatagtactaacatacgctctccatcaaaacaaaacgaaacaaaacaaactagcaaaa |
| SEQ ID NO: 23- | taggctgtccccagtgcaagtgcaggtgccagaacatttctctggcctaactggccggtacCAGC |
| XXX- SEQ ID NO. | CTGATTTCCCCGAAATGACGGCAGCCTGATTTCCCCGAAATG |
| 35 | ACGGCAGCCTGATTTCCCCGAAATGACGGCAGCCTGATTTCC |
| (“XXX” represents | CCGAAATGACGGCAGATCTACTAGAGGGTATATAATGGAAG |
| nucleotide sequence | CTCGACTTCCAGCTTGGCAATCCGGTACTGTTGGTAAAGCCA |
| encoding carrier | CC |
| protein of interest) | -XXX- |
| SEQ ID NO. 35 | AATTCTCACGGCTTCCCTCCCGAGGTGGAGGAGGAGGCCGCC |
| | GGCACCCTGCCCATGAGCTGCGCCCAGGAGAGCGGCATGGA |
| | TAGACACCCTGCTGCTTGCGCCAGCGCCAGGATCAACGTCTT |
| | CGAATTGGGAGGTGGCGGTAGCGGAGGTGGCGGTAGCCTCG |
| | AGAGCTCCAATTCACTGGCCGTCGTTTTACAACGTCGTGACT |
| | GGGAAAACCCTGGCGTTACCCAACTTAATCGCCTTGCAGCAC |
| | ATCCCCCTTTCGCCAGCTGGCGTAATAGCGAAGAGGCCCGCA |
| | CCGATCGCTAGTGAGGCCGGccgcttcgagcagacatgataagatacattgatgag |
| | tttggacaaaccacaactagaatgcagtgaaaaaaatgctttatttgtgaaatttgtgatgctattgctttat |
| | ttgtaaccattataagctgcaataaacaagttaacaacaacaattgcattcattttatgtttcaggttcagg |
| | gggaggtgtgggaggttttttaaagcaagtaaaacctetacaaatgtggtaaaatcgataaggatccgt |
| | ttgcgtattgggcgctcttccgctgatctgcgcagcaccatggcctgaaataacctctgaaagaggaac |
| | ttggttagctaccttctgaggcggaaagaaccagctgtggaatgtgtgtcagttagggtgtggaaagtc |
| | cccaggctccccagcaggcagaagtatgcaaagcatgcatctcaattagtcagcaaccaggtgtgga |
| | aagtccccaggctccccagcaggcagaagtatgcaaagcatgcatctcaattagtcagcaaccatagt |
| | cccgcccctaactccgcccatcccgcccctaactccgcccagttccgcccattctccgccccatggct |
| | gactaattttttttatttatgcagaggccgaggccgcctctgcctctgagctattccagaagtagtgagga |
| | ggcttttttggaggcctaggcttttgcaaaaagctcgattcttctgacactagcgccaccatgaagaagc |
| | ccgaactcaccgctaccagcgttgaaaaatttctcatcgagaagttcgacagtgtgagcgacctgatg |
| | cagttgtcggagggcgaagagagccgagccttcagcttcgatgtcggcggacgcggctatgtactg |
| | cgggtgaatagctgcgctgatggcttctacaaagaccgctacgtgtaccgccacttcgccagcgctgc |
| | actacccatccccgaagtgttggacatcggcgagttcagcgagagcctgacatactgcatcagtagac |
| | gcgcccaaggcgttactctccaagacctccccgaaacagagagcctgctgtgttacagcctgtcgcc |
| | gaagctatggatgctattgccgccgccgacctcagtcaaaccagcggcttcggcccattcgggcccc |
| | aaggcatcggccagtacacaacctggcgggatttcatttgcgccattgctgatccccatgtctaccact |
| | ggcagaccgtgatggacgacaccgtgtccgccagcgtagctcaagccctggacgaactgatgctgt |
| | gggccgaagactgtcccgaggtgcgccacctcgtccatgccgacttcggcagcaacaacgtcctga |
| | ccgacaacggccgcatcaccgccgtaatcgactggtccgaagctatgttcggggacagtcagtacga |
| | ggtggccaacatcttcttctggcggccctggctggcttgcatggagcagcagactcgctacttcgagc |
| | gccggcatcccgagctggccggcagccctcgtctgcgagcctacatgctgcgcatcggcctggatc |
| | agctctaccagagcctcgtggacggcaacttcgacgatgctgcctgggctcaaggccgctgcgatgc |
| | catcgtccgcagcggggccggcaccgtcggtcgcacacaaatcgctcgccggagcgcagccgtat |
| | ggaccgacggctgcgtcgaggtgaggccgacagcggcaaccgccggcccagtacacgaccgcg |
| | cgctaaggaggtaggtcgagtttaaactctagaaccggtcatggccgcaataaaatatctttattttcatt |
| | acatctgtgtgttggttttttgtgtgttcgaactagatgctgtcgaccgatgcccttgagagccttcaaccc |
| | agtcagctccttccggtgggcgcggggcatgactatcgtcgccgcacttatgactgtcttctttatcatg |
| | caactcgtaggacaggtgccggcagcgctcttccgcttcctcgctcactgactcgctgcgctcggtcg |
| | ttcggagcggcgagcggtatcagctcactcaaaggcggtaatacggttatccacagaatcagggga |
| | taacgcaggaaagaacatgtgagcaaaaggccagcaaaaggccaggaaccgtaaaaaggccgcg |
| | ttgctggcgtttttccataggctccgcccccctgacgagcatcacaaaaatcgacgctcaagtcagag |
| | gtggcgaaacccgacaggactataaagataccaggcgtttccccctggaagctccctcgtgcgctctc |
| | ctgttccgaccctgccgcttaccggatacctgtccgcctttctcccttcgggaagcgtggcgctttctcat |
| | agctcacgctgtaggtatctcagttcggtgtaggtcgttcgctccaagctgggctgtgtgcacgaaccc |
| | cccgttcagcccgaccgctgcgccttatccggtaactatcgtcttgagtccaacccggtaagacacga |
| | cttatcgccactggcagcagccactggtaacaggattagcagagcgaggtatgtaggcggtgctaca |
| | gagttcttgaagtggtggcctaactacggctacactagaagaacagtatttggtatctgcgctctgctga |
| | agccagttaccttcggaaaaagagttggtagctcttgatccggcaaacaaaccaccgctggtagcggt |
| | ggttttttttgtttgcaagcagcagattacgcgcagaaaaaaaggatctcaagaagatcctttgatcttttct |
| | acggggtctgacgctcagtggaacgaaaactcacgttaagggattttggtcatgagattatcaaaaag |
| | gatcttcacctagatccttttaaattaaaaatgaagttttaaatcaatctaaagtatatatgagtaaacttggt |
| | ctgacagcggccgcaaatgctaaaccactgcagtggttaccagtgcttgatcagtgaggcaccgatct |
| | cagcgatctgcctatttcgttcgtccatagtggcctgactccccgtcgtgtagatcactacgattcgtgag |
| | ggcttaccatcaggccccagcgcagcaatgatgccgcgagagccgcgttcaccggcccccgatttgt |
| | cagcaatgaaccagccagcagggagggccgagcgaagaagtggtcctgctactttgtccgcctcca |
| | tccagtctatgagctgctgtcgtgatgctagagtaagaagttcgccagtgagtagtttccgaagagttgt |
| | ggccattgctactggcatcgtggtatcacgctcgtcgttcggtatggcttcgttcaactctggttcccagc |
| | ggtcaagccgggtcacatgatcacccatattatgaagaaatgcagtcagctccttagggcctccgatc |
| | gttgtcagaagtaagttggccgcggtgttgtcgctcatggtaatggcagcactacacaattctcttaccg |
| | tcatgccatccgtaagatgcttttccgtgaccggcgagtactcaaccaagtcgttttgtgagtagtgtata |
| | cggcgaccaagctgctcttgcccggcgtctatacgggacaacaccgcgccacatagcagtactttga |
| | aagtgctcatcatcgggaatcgttcttcggggcggaaagactcaaggatcttgccgctattgagatcca |
| | gttcgatatagcccactcttgcacccagttgatcttcagcatcttttactttcaccagcgtttcggggtgtg |
| | caaaaacaggcaagcaaaatgccgcaaagaagggaatgagtgcgacacgaaaatgttggatgctca |
| | t |
| SEQ ID NO: 24 | actcgtcctttttcaatattattgaagcatttatcagggttactagtacgtctctcaaggataagtaagtaat |
| STAT3 Reporter | attaaggtacgggaggtattggacaggccgcaataaaatatctttattttcattacatctgtgtgttggtttt |
| Plasmid is SEQ ID | ttgtgtgaatcgatagtactaacatacgctctccatcaaaacaaaacgaaacaaaacaaactagcaaaa |
| NO: 24-XXX-SEQ | taggctgtccccagtgcaagtgcaggtgccagaacatttactggcctaactggccggtacctgagct |
| ID NO. 36 | cagcttcatttcccgtaaatcgtcgaagcttcatttcccgtaaatcgtcgaagcttcatttcccgtaaatcg |
| (“XXX” represents | tcgaagcttcatttcccgtaaatcgtcgaagcttcatttcccgtaaatcgtcgactcgaggatatcaaGA |
| nucleotide sequence | TCTACTAGAGGGTATATAATGGAAGCTCGACTTCCAGCTTGG |
| encoding carrier | CAATCCGGTACTGTTGGTAAAGCCACC |
| protein of interest) | -XXX- |
| SEQ ID NO. 36 | AAATTCTCACGGCTTCCCTCCCGAGGTGGAGGAGCAGGCCGC |
| | CGGCACCCTGCCCATGAGCTGCGCCCAGGAGAGCGGCATGG |
| | ATAGACACCCTGCTGCTTGCGCCAGCGCCAGGATCAACGTCT |
| | TCGAATTGGGAGGTGGCGGTAGCGGAGGTGGCGGTAGCCTC |
| | GAGAGCTCCAATTCACTGGCCGTCGTTTTACAACGTCGTGAC |
| | TGGGAAAACCCTGGCGTTACCCAACTTAATCGCCTTGCAGCA |
| | CATCCCCCTTTCGCCAGCTGGCGTAATAGCGAAGAGGCCCGC |
| | ACCGATCGCTAGTGAGGCCGGccgcttcgagcagacatgataagatacattgatg |
| | agtttggacaaaccacaactagaatgcagtgaaaaaaatgctttatttgtgaaatttgtgatgctattgctt |
| | tatttgtaaccattataagctgcaataaacaagttaacaacaacaattgcattcattttatgtttcaggttca |
| | gggggaggtgtgggaggttttttaaagcaagtaaaacctctacaaatgtggtaaaatcgataaggatcc |
| | gtttgcgtattgggcgctcttccgctgatctgcgcagcaccatggcctgaaataacctctgaaagagga |
| | acttggttagctaccttctgaggcggaaagaaccagctgtggaatgtgtgtcagttagggtgtggaaa |
| | gtccccaggctccccagcaggcagaagtatgcaaagcatgcatctcaattagtcagcaaccaggtgt |
| | ggaaagtccccaggctccccagcaggcagaagtatgcaaagcatgcatctcaattagtcagcaacca |
| | tagtcccgcccctaactccgcccatcccgcccctaactccgcccagttccgcccattctccgccccatg |
| | gctgactaattttttttatttatgcagaggccgaggccgcctctgcctctgagctattccagaagtagtga |
| | ggaggcttttttggaggcctaggcttttgcaaaaagctcgattcttctgacactagcgccaccatgaaga |
| | agcccgaactcaccgctaccagcgttgaaaaatttctcatcgagaagttcgacagtgtgagcacctg |
| | atgcagttgtcggagggcgaagagagccgagccttcagcttcgatgtcggcggacgcggctatgta |
| | ctgcgggtgaatagctgcgctgatggcttctacaaagaccgctacgtgtaccgccacttcgccagcgc |
| | tgcactacccatccecgaagtgttggacatcggcgagttcagcgagagcctgacatactgcatcagta |
| | gacgcgcccaaggcgttactctccaagacctccccgaaacagagctgcctgctgtgttacagcctgtc |
| | gccgaagctatggatgctattgccgccgccgacctcagtcaaaccagcggcttcggcccattcgggc |
| | cccaaggcatcggccagtacacaacctggcgggatttcatttgcgccattgctgatccccatgtctacc |
| | actggcagaccgtgatggacgacaccgtgtccgccagcgtagctcaagccctggacgaactgatgc |
| | tgtgggccgaagactgtcccgaggtgcgccacctcgtccatgccgacttcggcagcaacaacgtcct |
| | gaccgacaacggccgcatcaccgccgtaatcgactggtccgaagctatgttcggggacagtcagtac |
| | gaggtggccaacatcttcttctggcggccctggctggcttgcatggagcagcagactcgctacttcga |
| | gcgccggcatcccgagctggccggcagccctcgtctgcgagcctacatgagcgcatcggcctgga |
| | tcagctctaccagagcctcgtggacggcaacttcgacgatgctgcctgggctcaaggccgctgcgat |
| | gccatcgtccgcagcggggccggcaccgtcggtcgcacacaaatcgctcgccggagcgcagccgt |
| | atggaccgacggctgcgtcgaggtgctggccgacagcggcaaccgccggcccagtacacgaccg |
| | cgcgctaaggaggtaggtcgagtttaaactctagaaccggtcatggccgcaataaaatatctttattttc |
| | attacatctgtgtgttggttttttgtgtgttcgaactagatgctgtcgaccgatgcccttgagagccttcaac |
| | ccagtcagctccttccggtgggcgcggggcatgactatcgtcgccgcacttatgactgtcttctttatca |
| | tgcaactcgtaggacaggtgccggcagcgctcttccgcttcctcgctcactgactcgctgcgctcggt |
| | cgttcggctgcggcgagcggtatcagctcactcaaaggcggtaatacggttatccacagaatcaggg |
| | gataacgcaggaaagaacatgtgagcaaaaggccagcaaaaggccaggaaccgtaaaaaggccg |
| | cgttgctggcgtttttccataggctccgcccccctgacgagcatcacaaaaatcgacgctcaagtcaga |
| | ggtggcgaaacccgacaggactataaagataccaggcgtttccccctggaagctccctcgtgcgctc |
| | tcctgttccgaccctgccgcttaccggatacctgtccgcctttctcccttcgggaagcgtggcgctttctc |
| | atagctcacgctgtaggtatctcagttcggtgtaggtcgttcgctccaagctgggctgtgtgcacgaac |
| | cccccgttcagcccgaccgctgcgccttatccggtaactatcgtcttgagtccaacccggtaagacac |
| | gacttatcgccactggcagcagccactggtaacaggattagcagagcgaggtatgtaggcggtgcta |
| | cagagttatgaagtggtggcctaactacggctacactagaagaacagtatttggtatctgcgactgct |
| | gaagccagttaccttcggaaaaagagttggtagctcttgatccggcaaacaaaccaccgctggtagc |
| | ggtggtttttttgtttgcaagcagcagattacgcgcagaaaaaaaggatctcaagaagatcctttgatctt |
| | ttctacggggtctgacgctcagtggaacgaaaactcacgttaagggattttggtcatgagattatcaaaa |
| | aggatcttcacctagatccttttaaattaaaaatgaagttttaaatcaatctaaagtatatatgagtaaactt |
| | ggtctgacagcggccgcaaatgctaaaccactgcagtggttaccagtgcttgatcagtgaggcaccg |
| | atacagcgatctgcctatttcgttcgtccatagtggcctgactccccgtcgtgtagatcactacgattcgt |
| | gagggcttaccatcaggccccagcgcagcaatgatgccgcgagagccgcgttcaccggcccccga |
| | tttgtcagcaatgaaccagccagcagggagggccgagcgaagaagtggtcctgctactttgtccgcc |
| | tccatccagtctatgagcgctgtcgtgatgctagagtaagaagttcgccagtgagtagtttccgaaga |
| | gttgtggccattgctactggcatcgtggtatcacgctcgtcgttcggtatggcttcgttcaactctggttcc |
| | cagcggtcaagccgggtcacatgatcacccatattatgaagaaatgcagtcagctccttagggcctcc |
| | gatcgttgtcagaagtaagttggccgcggtgttgtcgctcatggtaatggcagcactacacaattctctt |
| | accgtcatgccatccgtaagatgcttttccgtgaccggcgagtactcaaccaagtcgttttgtgagtagt |
| | gtatacggcgaccaagctgctcttgcccggcgtctatacgggacaacaccgcgccacatagcagtac |
| | tttgaaagtgctcatcatcgggaatcgttcttcggggcggaaagactcaaggatcttgccgctattgag |
| | atccagttcgatatagcccactcttgcacccagttgatcttcagcatcttttactttcaccagcgtttcggg |
| | gtgtgcaaaaacaggcaagcaaaatgccgcaaagaagggaatgagtgcgacacgaaaatgttggat |
| | gctcat |
| SEQ ID NO: 25 | actcgtcctttttcaatattattgaagcatttatcagggttactagtacgtctctcaaggataagtaagtaat |
| STAT5 Reporter | attaaggtacgggaggtattggacaggccgcaataaaatatctttattttcattacatctgtgtgttggtttt |
| Plasmid is SEQ ID | ttgtgtgaatcgatagtactaacatacgctctccatcaaaacaaaacgaaacaaaacaaactagcaaaa |
| NO: 25-XXX -SEQ | taggctgtccccagtgcaagtgcaggtgccagaacatttctaggcctaacTGGCCGGTACct |
| ID NO. 37 | gagctcagttctgagaaaagtagttctgagaaaagtagttctgagaaaagtagttctgagaaaagtagtt |
| (“XXX” represents | ctgagaaaagtctcgaggatatcaaGATCTACTAGAGGGTATATAATGGAA |
| nucleotide sequence | GCTCGACTTCCAGCTTGGCAATCCGGTACTGTTGGTAAAGCC |
| encoding carrier | ACC |
| protein of interest) | -XXX- |
| SEQ ID NO. 37 | AAATTCTCACGGCTTCCCTCCCGAGGTGGAGGAGCAGGCCGC |
| | CGGCACCCTGCCCATGAGCTGCGCCCAGGAGAGCGGCATGG |
| | ATAGACACCCTGCTGCTTGCGCCAGCGCCAGGATCAACGTCT |
| | TCGAATTGGGAGGTGGCGGTAGCGGAGGTGGCGGTAGCCTC |
| | GAGAGCTCCAATTCACTGGCCGTCGTTTTACAACGTCGTGAC |
| | TGGGAAAACCCTGGCGTTACCCAACTTAATCGCCTTGCAGCA |
| | CATCCCCCTTTCGCCAGCTGGCGTAATAGCGAAGAGGCCCGC |
| | ACCGATCGCTAGTGAGGCCGGccgcttcgagcagacatgataagatacattgatg |
| | agtttggacaaaccacaactagaatgcagtgaaaaaaatgctttatttgtgaaatttgtgatgctattgctt |
| | tatttgtaaccattataagctgcaataaacaagttaacaacaacaattgcattcattttatgtttcaggttca |
| | gggggaggtgtgggaggttttttaaagcaagtaaaacctctacaaatgtggtaaaatcgataaggatcc |
| | gtttgcgtattgggcgctcttccgctgatctgcgcagcaccatggcctgaaataacctctgaaagagga |
| | acttggttagctaccttctgaggcggaaagaaccagctgtggaatgtgtgtcagttagggtgtggaaa |
| | gtccccaggctccccagcaggcagaagtatgcaaagcatgcatctcaattagtcagcaaccaggtgt |
| | ggaaagtccccaggctccccagcaggcagaagtatgcaaagcatgcatctcaattagtcagcaacca |
| | tagtcccgcccctaactccgcccatcccgcccctaactccgcccagttccgcccattaccgccccatg |
| | gctgactaattttttttatttatgcagaggccgaggccgcctctgcctctgagctattccagaagtagtga |
| | ggaggcttttttggaggcctaggcttttgcaaaaagctcgattcttctgacactagcgccaccatgaaga |
| | agcccgaactcaccgctaccagcgttgaaaaatttctcatcgagaagttcgacagtgtgagcgacctg |
| | atgcagttgtcggagggcgaagagagccgagccttcagcttcgatgtcggcggacgcggctatgta |
| | ctgcgggtgaatagctgcgctgatggcttctacaaagaccgctacgtgtaccgccacttcgccagcgc |
| | tgcactacccatccccgaagtgttggacatcggcgagttcagcgagagcctgacatactgcatcagta |
| | gacgcgcccaaggcgttactctccaagacctccccgaaacagagctgcctgctgtgttacagcctgtc |
| | gccgaagctatggatgctattgccgccgccgacctcagtcaaaccagcggcttcggcccattcgggc |
| | cccaaggcatcggccagtacacaacctggcgggatttcatttgcgccattgctgatccccatgtctacc |
| | actggcagaccgtgatggacgacaccgtgtccgccagcgtagctcaagccctggacgaactgatgc |
| | tgtgggccgaagactgtcccgaggtgcgccacctcgtccatgccgacttcggcagcaacagtcct |
| | gaccgacaacggccgcatcaccgccgtaatcgactggtccgaagctatgttcggggacagtcagtac |
| | gaggtggccaacatcttcttctggcggccctggctggcttgcatggagcagcagactcgctacttcga |
| | gcgccggcatcccgagctggccggcagccctcgtctgcgagcctacatgctgcgcatcggcctgga |
| | tcagctctaccagagcctcgtggacggcaacttcgacgatgctgcctgggctcaaggccgctgcgat |
| | gccatcgtccgcagcggggccggcaccgtcggtcgcacacaaatcgctcgccggagcgcagccgt |
| | atggaccgacggctgcgtcgaggtgctggccgacagcggcaaccgccggcccagtacacgaccg |
| | cgcgctaaggaggtaggtcgagtttaaaactagaaccggtcatggccgcaataaaatatctttattttc |
| | attacatctgtgtgttggttttttgtgtgttcgaactagatgctgtcgaccgatgcccttgagagccttcaac |
| | ccagtcagctccttccggtgggcgcggggcatgactatcgtcgccgcacttatgactgtcttctttatca |
| | tgcaactcgtaggacaggtgccggcagcgctatccgcttcctcgctcactgactcgctgcgctcggt |
| | cgttcggctgcggcgagcggtatcagctcactcaaaggcggtaatacggttatccacagaatcaggg |
| | gataacgcaggaaagaacatgtgagcaaaaggccagcaaaaggccaggaaccgtaaaaaggccg |
| | cgttgctggcgtttttccataggctccgcccccctgacgagcatcacaaaaatcgacgctcaagtcaga |
| | ggtggcgaaacccgacaggactataaagataccaggcgtttccccctggaagctccctcgtgcgctc |
| | tcctgttccgaccctgccgcttaccggatacctgtccgcctttctcccttcgggaagcgtggcgctttctc |
| | atagctcacgctgtaggtatctcagttcggtgtaggttcgttcgctccaagctgggctgtgtgcacgaac |
| | cccccgttcagcccgaccgctgcgccttatccggtaactatcgtcttgagtccaacccggtaagacac |
| | gacttatcgccactggcagcagccactggtaacaggattagcagagcgaggtatgtaggcggtgcta |
| | cagagttcttgaagtggtggcctaactacggctacactagaagaacagtatttggtatctgcgctctgct |
| | gaagccagttaccttcggaaaaagagttggtagctcttgatccggcaaacaaaccaccgaggtagc |
| | ggtggtttttttgtttgcaagcagcagattacgcgcagaaaaaaaggatctcaagaagatcctttgatctt |
| | ttctacggggtctgacgctcagtggaacgaaaactcacgttaagggattttggtcatgagattatcaaaa |
| | aggatcttcacctagatccttttaaattaaaaatgaagttttaaatcaatctaaagtatatatgagtaaactt |
| | ggtctgacagcggccgcaaatgctaaaccactgcagtggttaccagtgcttgatcagtgaggcaccg |
| | atctcagcgatctgcctatttcgttcgtccatagtggcctgactccccgtcgtgtagatcactacgattcgt |
| | gagggcttaccatcaggccccagcgcagcaatgatgccgcgagagccgcgttcaccggcccccga |
| | tttgtcagcaatgaaccagccagcagggagggccgagcgaagaagtggtcctgctactttgtccgcc |
| | tccatccagtctatgagagctgtcgtgatgctagagtaagaagttcgccagtgagtagtttccgaaga |
| | gttgtggccattgctactggcatcgtggtatcacgctcgtcgttcggtatggcttcgttcaactctggttcc |
| | cagcggtcaagccgggtcacatgatcacccatattatgaagaaatgcagtcagaccttagggcctcc |
| | gatcgttgtcagaagtaagttggccgcggtgttgtcgctcatggtaatggcagcactacacaattctctt |
| | accgtcatgccatccgtaagatgcttttccgtgaccggcgagtactcaaccaagtcgttttgtgagtagt |
| | gtatacggcgaccaagctgctcttgcccggcgtctatacgggacaacaccgcgccacatagcagtac |
| | tttgaaagtgctcatcatcgggaatcgttcttcggggcggaaagactcaaggatcttgccgctattgag |
| | atccagttcgatatagcccactcttgcacccagttgatcttcagcatcttttactttcaccagcgtttcggg |
| | gtgtgcaaaaacaggcaagcaaaatgccgcaaagaagggaatgagtgcgacacgaaaatgttggat |
| | gctcat |
| SEQ ID NO: 26 | actcgtcctttttcaatattattgaagcatttatcagggttactagtacgtctctcaaggataagtaagtaat |
| RORgammaT | attaaggtacgggaggtattggacaggccgcaataaaatatctttattacattacatctgtgtgttggtttt |
| Reporter Plasmid is | ttgtgtgaatcgatagtactaacatacgctctccatcaaaacaaaacgaaacaaaacaaactagcaaaa |
| SEQ ID NO: 26- | taggctgtccccagtgcaagtgcaggtgccagaacatttctctggcctaacTGGCCGGTACct |
| XXX-SEQ ID NO. | gagctcGGTAAGTAGGTCATGGTAAGTAGGTCATGGTAAGTAGG |
| 38 | TCATGGTAAGTAGGTCATGGTAAGTAGGTCATCGTGACctcgag |
| (“XXX” represents | gatatcaaGATCTACTAGAGGGTATATAATGGAAGCTCGACTTCC |
| nucleotide sequence | AGCTTGGCAATCCGGTACTGTTGGTAAAGCCACC |
| encoding carrier | -XXX- |
| protein of interest) | |
| SEQ ID NO. 38 | AAATTCTCACGGCTTCCCTCCCGAGGTGGAGGAGCAGGCCGC |
| | CGGCACCCTGCCCATGAGCTGCGCCCAGGAGAGCGGCATGG |
| | ATAGACACCCTGCTGCTTGCGCCAGCGCCAGGATCAACGTCT |
| | TCGAATTGGGAGGTGGCGGTAGCGGAGGTGGCGGTAGCCTC |
| | GAGAGCTCCAATTCACTGGCCGTCGTTTTACAACGTCGTGAC |
| | TGGGAAAACCCTGGCGTTACCCAACTTAATCGCCTTGCAGCA |
| | CATCCCCCTTTCGCCAGCTGGCGTAATAGCGAAGAGGCCCGC |
| | ACCGATCGCTAGTGAGGCCGGccgcttcgagcagacatgataagatacattgatg |
| | agtttggacaaaccacaactagaatgcagtgaaaaaaatgctttatttgtgaaatttgtgatgctattgctt |
| | tatttgtaaccattataagctgcaataaacaagttaacaacaacaattgcattcattttatgtttcaggttca |
| | gggggaggtgtgggaggttttttaaagcaagtaaaacctctacaaatgtggtaaaatcgataaggatcc |
| | gtttgcgtattgggcgacttccgctgatctgcgcagcaccatggcctgaaataacctagaaagagga |
| | acttggttagctaccttctgaggcggaaagaaccagagtggaatgtgtgtcagttagggtgtggaaa |
| | gtccccaggctccccagcaggcagaagtatgcaaagcatgcatctcaattagtcagcaaccaggtgt |
| | ggaaagtccccaggctccccagcaggcagaagtatgcaaagcatgcatctcaattagtcagcaacca |
| | tagtcccgcccctaactccgcccatcccgcccctaactccgcccagttccgcccattctccgccccatg |
| | gctgactaattttttttatttatgcagaggccgaggccgcctctgcctctgagctattccagaagtagtga |
| | ggaggcttttttggaggcctaggcttttgcaaaaagctcgattcttctgacactagcgccaccatgaaga |
| | agcccgaactcaccgctaccagcgttgaaaaatttctcatcgagaagttcgacagtgtgagcgacctg |
| | atgcagttgtcggagggcgaagagagccgagccttcagcttcgatgtcggcggacgcggctatgta |
| | ctgcgggtgaatagctgcgctgatggcttctacaaagaccgctacgtgtaccgccacttcgccagcgc |
| | tgcactacccatccccgaagtgttggacatcggcgagttcagcgagagcctgacatactgcatcagta |
| | gacgcgcccaaggcgttactctccaagacctccccgaaacagagctgcctgctgtgttacagcctgtc |
| | gccgaagctatggatgctattgccgccgccgacctcagtcaaaccagcggcttcggcccattcgggc |
| | cccaaggcatcggccagtacacaacctggcgggatttcatttgcgccattgctgatccccatgtctacc |
| | actggcagaccgtgatggacgacaccgtgtccgccagcgtagctcaagccaggacgaactgatgc |
| | tgtgggccgaagactgtcccgaggtgcgccacctcgtccatgccgacttcggcagcaacaacgtcct |
| | gaccgacaacggccgcatcaccgccgtaatcgactggtccgaagctatgttcggggacagtcagtac |
| | gaggtggccaacatcttcttctggcggccctggctggcttgcatggagcagcagactcgctacttcga |
| | gcgccggcatcccgagctggccggcagccctcgtctgcgagcctacatgctgcgcatcggcctgga |
| | tcagctctaccagagcctcgtggacggcaacttcgacgatgctgcctgggctcaaggccgctgcgat |
| | gccatcgtccgcagcggggccggcaccgtcggtcgcacacaaatcgctcgccggagcgcagccgt |
| | atggaccgacggctgcgtcgaggtgctggccgacagcggcaaccgccggcccagtacacgaccg |
| | cgcgctaaggaggtaggtcgagtttaaactctagaaccggtcatggccgcaataaaatatctttattttc |
| | attacatctgtgtgttggttttttgtgtgttcgaactagatgctgtcgaccgatgcccttgagagccttcaac |
| | ccagtcagaccttccggtgggcgcggggcatgactatcgtcgccgcacttatgactgtcttctttatca |
| | tgcaactcgtaggacaggtgccggcagcgctcttccgcttcctcgctcactgactcgctgcgctcgg |
| | cgttcggctgcggcgagcggtatcagctcactcaaaggcggtaatacggttatccacagaatcaggg |
| | gataacgcaggaaagaacatgtgagcaaaaggccagcaaaaggccaggaaccgtaaaaaggccg |
| | cgttgctggcgtttttccataggctccgcccccctgacgagcatcacaaaaatcgacgctcaagtcaga |
| | ggtggcgaaacccgacaggactataaagataccaggcgtttccccctggaagaccctcgtgcgctc |
| | tcctgttccgaccctgccgcttaccggatacctgtccgcctttctcccttcgggaagcgtggcgctttctc |
| | atagctcacgctgtaggtatctcagttcggtgtaggtcgttcgctccaagctgggctgtgtgcacgaac |
| | cccccgttcagcccgaccgctgcgccttatccggtaactatcgtcttgagtccaacccggtaagacac |
| | gacttatcgccactggcagcagccactggtaacaggattagcagagcgaggtatgtaggcggtgcta |
| | cagagttcttgaagtggtggcctaactacggctacactagaagaacagtatttggtatctgcgctctgct |
| | gaagccagttaccttcggaaaaagagttggtagctcttgatccggcaaacaaaccaccgctggtagc |
| | ggtggtttttttgtttgcaagcagcagattacgcgcagaaaaaaaggatctcaagaagatcctttgatctt |
| | ttctacggggtctgacgctcagtggaacgaaaactcacgttaagggattttggtcatgagattatcaaaa |
| | aggatcttcacctagatccttttaaattaaaaatgaagttttaaatcaatctaaagtatatatgagtaaactt |
| | ggtctgacagcggccgcaaatgctaaaccactgcagtggttaccagtgcttgatcagtgaggcaccg |
| | atctcagcgatctgcctatttcgttcgtccatagtggcctgactccccgtcgtgtagatcactacgattcgt |
| | gagggcttaccatcaggccccagcgcagcaatgatgccgcgagagccgcgttcaccggcccccga |
| | tttgtcagcaatgaaccagccagcagggagggccgagcgaagaagtggtcctgctactttgtccgcc |
| | tccatccagtctatgagctgagtcgtgatgctagagtaagaagttcgccagtgagtagtttccgaaga |
| | gttgtggccattgctactggcatcgtggtatcacgctcgtcgttcggtatggcttcgttcaactctggttcc |
| | cagcggtcaagccgggtcacatgatcacccatattatgaagaaatgcagtcagctccttagggcacc |
| | gatcgttgtcagaagtaagttggccgcggtgttgtcgctcatggtaatggcagcactacacaattctctt |
| | accgtcatgccatccgtaagatgcttttccgtgaccggcgagtactcaaccaagtcgttttgtgagtagt |
| | gtatacggcgaccaagctgctcttgcccggcgtctatacgggacaacaccgcgccacatagcagtac |
| | tttgaaagtgctcatcatcgggaatcgttcttcggggcggaaagactcaaggatcttgccgctattgag |
| | atccagttcgatatagcccactcttgcacccagttgatatcagcatcttttactttcaccagcgtttcggg |
| | gtgtgcaaaaacaggcaagcaaaatgccgcaaagaagggaatgagtgcgacacgaaaatgttggat |
| | gctcat |
| SEQ ID NO: 27 | actcgtcctttttcaatattattgaagcatttatcagggttactagtacgtactcaaggataagtaagtaat |
| 5xGal4UAS | attaaggtacgggaggtattggacaggccgcaataaaatatctttattttcattacatctgtgtgttggtttt |
| Reporter Plasmid is | ttgtgtgaatcgatagtactaacatacgctaccatcaaaacaaaacgaaacaaaacaaactagcaaaa |
| SEQ ID NO: 27- | taggctgtccccagtgcaagtgcaggtgccagaacatttctctggcctaactggccggtaccAAG |
| XXX-SEQ ID NO. | ATTTctgcagCGGAGTACTGTCCTCCGAGCGGAGTACTGTCCTCC |
| 39 | GAGCGGAGTACTGTCCTCCGAGCGGAGTACTGTCCTCCGAGC |
| (“XXX” represents | GGAGTACTGTCCTCCGCTCGAGGATATCaGATCTACTAGAGG |
| nucleotide sequence | GTATATAATGGAAGCTCGACTTCCAGCTTGGCAATCCGGTAC |
| encoding carrier | TGTTGGTAAAGCCACC |
| protein of interest) | -XXX- |
| SEQ ID NO. 39 | AAATTCTCACGGCTTCCCTCCCGAGGTGGAGGAGCAGGCCGC |
| | CGGCACCCTGCCCATGAGCTGCGCCCAGGAGAGCGGCATGG |
| | ATAGACACCCTGCTGCTTGCGCCAGCGCCAGGATCAACGTCT |
| | TCGAATTGGGAGGTGGCGGTAGCGGAGGTGGCGGTAGCCTC |
| | GAGAGCTCCAATTCACTGGCCGTCGTTTTACAACGTCGTGAC |
| | TGGGAAAACCCTGGCGTTACCCAACTTAATCGCCTTGCAGCA |
| | CATCCCCCTTTCGCCAGCTGGCGTAATAGCGAAGAGGCCCGC |
| | ACCGATCGCTAGTGAGGCCGGccgcttcgagcagacatgataagatacattgatg |
| | agtttggacaaaccacaactagaatgcagtgaaaaaaatgctttatttgtgaaatttgtgatgctattgctt |
| | tatttgtaaccattataagctgcaataaacaagttaacaacaacaattgcattcattttatgtttcaggttca |
| | gggggaggtgtgggaggttttttaaagcaagtaaaacctctacaaatgtggtaaaatcgataaggatcc |
| | gtttgcgtattgggcgctcttccgctgatctgcgcagcaccatggcctgaaataacctctgaaagagga |
| | acttggttagctaccttctgaggcggaaagaaccagagtggaatgtgtgtcagttagggtgtggaaa |
| | gtccccaggctccccagcaggcagaagtatgcaaagcatgcatctcaattagtcagcaaccaggtgt |
| | ggaaagtccccaggctccccagcaggcagaagtatgcaaagcatgcatctcaattagtcagcaacca |
| | tagtcccgcccctaactccgcccatcccgcccctaactccgcccagttccgcccattctccgccccatg |
| | gctgactaattttattatttatgcagaggccgaggccgcctctgcctctgagctattccagaagtagtga |
| | ggaggctttttttggaggcctaggcttttgcaaaaagctcgattcttctgacactagcgccaccatgaaga |
| | agcccgaactcaccgctaccagcgttgaaaaatttctcatcgagaagttcgacagtgtgagcgacctg |
| | atgcagttgtcggagggcgaagagagccgagccttcagcttcgatgtcggcggacgcggctatgta |
| | ctgcgggtgaatagctgcgctgatggcttctacaaagaccgctacgtgtaccgccacttcgccagcgc |
| | tgcactacccatccccgaagtgttggacatcggcgagttcagcgagagcctgacatactgcatcagta |
| | gacgcgcccaaggcgttactctccaagacctccccgaaacagagctgcctgctgtgttacagcctgtc |
| | gccgaagctatggatgctattgccgccgccgacctcagtcaaaccagcggcttcggcccattcgggc |
| | cccaaggcatcggccagtacacaacctggcgggatttcatttgcgccattgagatccccatgtctacc |
| | actggcagaccgtgatggacgacaccgtgtccgccagcgtagctcaagccctggacgaactgatgc |
| | tgtgggccgaagactgtcccgaggtgcgccacctcgtccatgccgacttcggcagcaacaacgtcct |
| | gaccgacaacggccgcatcaccgccgtaatcgactggtccgaagctatgttcggggacagtcagtac |
| | gaggtggccaacatcttcttctggcggccctggctggcttgcatggagcagcagactcgctacttcga |
| | gcgccggcatcccgagtggccggcagccctcgtctgcgagcctacatgctgcgcatcggcctgga |
| | tcagactaccagagcctcgtggacggcaacttcgacgatgagcctgggctcaaggccgctgcgat |
| | gccatcgtccgcagcggggccggcaccgtcggtcgcacacaaatcgctcgccggagcgcagccgt |
| | atggaccgacggctgcgtcgaggtgctggccgacagcggcaaccgccggcccagtacacgaccg |
| | cgcgctaaggaggtaggtcgagtttaaactctagaaccggtcatggccgcaataaaatatctttattttc |
| | attacatctgtgtgttggttttttgtgtgttcgaactagatgctgtcgaccgatgcccttgagagccttcaac |
| | ccagtcagctccttccggtgggcgcggggcatgactatcgtcgccgcacttatgactgtcttctttatca |
| | tgcaactcgtaggacaggtgccggcagcgctcttccgcttcctcgctcactgactcgctgcgctcggt |
| | cgttcggctgcggcgagcggtatcagctcactcaaaggcggtaatacggttatccacagaatcaggg |
| | gataacgcaggaaagaacatgtgagcaaaaggccagcaaaaggccaggaaccgtaaaaaggccg |
| | cgttgctggcgtttttccataggctccgcccccctgacgagcatcacaaaaatcgacgctcaagtcaga |
| | ggtggcgaaacccgacaggactataaagataccaggcgtttccccctggaagctccctcgtgcgctc |
| | tcctgttccgaccctgccgcttaccggatacctgtccgcctttctcccttcgggaagcgtggcgctttctc |
| | atagctcacgctgtaggtatctcagttcggtgtaggtcgttcgctccaagctgggctgtgtgcacgaac |
| | cccccgttcagcccgaccgctgcgccttatccggtaactatcgtcttgagtccaacccggtaagacac |
| | gacttatcgccactggcagcagccactggtaacaggattagcagagcgaggtatgtaggcggtgcta |
| | cagagttcttgaagtggtggcctaactacggctacactagaagaacagtatttggtatctgcgctctgct |
| | gaagccagttaccttcggaaaaagagttggtagacttgatccggcaaacaaaccaccgctggtagc |
| | ggtggtttttttgtttgcaagcagcagattacgcgcagaaaaaaaggatctcaagaagatcctttgatctt |
| | ttctacggggtctgacgctcagtggaacgaaaactcacgttaagggattttggtcatgagattatcaaaa |
| | aggatcttcacctagatccttttaaattaaaaatgaagttttaaatcaatctaaagtatatatgagtaaactt |
| | ggtctgacagcggccgcaaatgctaaaccactgcagtggttaccagtgcttgatcagtgaggcaccg |
| | atctcagcgatctgcctatttcgttcgtccatagtggcctgactccccgtcgtgtagatcactacgattcgt |
| | gagggcttaccatcaggccccagcgcagcaatgatgccgcgagagccgcgttcaccggcccccga |
| | tttgtcagcaatgaaccagccagcagggagggccgagcgaagaagtggtcctgctactttgtccgcc |
| | tccatccagtctatgagctgctgtcgtgatgctagagtaagaagttcgccagtgagtagtttccgaaga |
| | gttgtggccattgctactggcatcgtggtatcacgctcgtcgttcggtatggcttcgttcaactctggttcc |
| | cagcggtcaagccgggtcacatgatcacccatattatgaagaaatgcagtcagctccttagggcctcc |
| | gatcgttgtcagaagtaagttggccgcggtgttgtcgctcatggtaatggcagcactacacaattctctt |
| | accgtcatgccatccgtaagatgcttttccgtgaccggcgagtactcaaccaagtcgttttgtgagtagt |
| | gtatacggcgaccaagctgctcttgcccggcgtctatacgggacaacaccgcgccacatagcagtac |
| | tttgaaagtgctcatcatcgggaatcgttcttcggggcggaaagactcaaggatcttgccgctattgag |
| | atccagttcgatatagcccactcttgcacccagttgatcttcagcatcttttactttcaccagcgtttcggg |
| | gtgtgcaaaaacaggcaagcaaaatgccgcaaagaagggaatgagtgcgacacgaaaatgttggat |
| | gctcat |

In certain embodiments, the nucleic acid is a chromosome of the cell. For example, a chromosome of the cell may be modified (e.g., using a genome editing technology such as homologous recombination, CRISPR-Cas9, transcription activator-like effector nucleases (TALEN), and/or the like) such that the region encoding the ED is inserted into the chromosome. In some embodiments, the region encoding the ED is inserted into the genome of the cell such that the region encoding the ED is operably coupled to a native promoter region of the chromosome. The native promoter region may be a promoter region that finds use for assessing transcriptional activation of one or more genes of interest, and/or one or more cell signaling pathways of interest. As just one example, if one wishes to assess activation of an NFκB signaling pathway in the cell, the region encoding the ED may be inserted site-specifically downstream of a promoter region that includes an NFκB binding site. In certain embodiments, the region encoding the ED is inserted into a chromosome along with a promoter region (that is—an exogenous promoter region), where the region that encodes the ED is operably coupled to the exogenous promoter region. In any of the embodiments in which the nucleic acid is a chromosome of the cell, the chromosome may be a nuclear chromosome or a mitochondrial chromosome.

In certain embodiments, the nucleic acid further encodes a carrier protein fused to the ED, such that ED-carrier protein fusions are expressed when the promoter region is active. The carrier protein chosen for the ED may confer different desired physical or biological properties to ED (e.g. stability, localization, biological inertness, detection by another method distinct from EFC, etc). According to some embodiments, the carrier protein includes a domain selected to affect the stability of the ED-carrier protein fusions. In certain embodiments, the domain is selected to increase the stability of the ED-carrier protein fusions as compared to ED-carrier protein fusions lacking the domain. In other embodiments, the domain is selected to destabilize the ED-carrier protein fusions as compared to ED-carrier protein fusions lacking the domain. For example, the domain may be a domain that targets the ED-carrier protein fusions for proteasomal degradation (e.g., ubiquitin-dependent proteasomal degradation). One example of a domain that may be employed to target the ED-carrier protein fusions for proteasomal degradation a proline (P), glutamic acid (E), serine (S) and threonine (T) (PEST) degradation signal. Another example of such a domain is a CL1 degradation signal. The amino acid sequences of example PEST and CL1 degradation signals are provided in Table 2 below.

TABLE 2
Degradation Signal Amino Acid Sequences

| | |
|---|---|
| PEST degradation | SHGFPPEVEEQAAGTLPMSCAQESGMDRHPA |
| signal | ACASARINV |
| (SEQ ID NO: 28) | |
| CL1 degradation | ACKNWFSSLSHFVIHL |
| signal | |
| (SEQ ID NO: 29) | |

In certain embodiments, the carrier protein includes two or more domains selected to affect, in combination, the stability of the ED-carrier protein fusions. For example, a carrier protein could include a PEST degradation signal and a CL1 degradation signal to enhance the targeting of the ED-carrier protein fusions for proteasomal degradation relative to the targeting achieved using a single such signal.

In many embodiments, the promoter is further coupled with a carrier protein such that the presence of carrier protein enhances the signal resulting in a more sensitive and potent assay as compared to existing reporter systems which rely on expression of 1) full length (single polypeptide) enzymes such as full-length luciferase, β-galactosidase, chloramphenicol acetyl transferase (CAT); and 2) fluorescent proteins. The carrier protein may be operably coupled to a promoter region. The carrier protein may be one with a detectable activity such that the expression of carrier protein can be detected by a known detection method. In many other embodiments, the detectable activity of the carrier protein is not same as the enzymatic activity of the β-galactosidase enzyme.

In further embodiments, a carrier protein may is fused with the ED enzyme fragment of β-galactosidase enzyme which is operably coupled to a promoter region. In many embodiments, the carrier protein may co-express with the ED enzyme fragment when the promoter region is active resulting in an enhanced output signal or data points. A carrier protein as used in the present disclosed method may possess a detectable enzymatic activity which is not same as the enzymatic activity of β-galactosidase wherein the carrier protein enzymatic activity can be detected when a promoter region is active using known detection methods for the said enzymatic activity. The carrier protein with detectable enzymatic activity may be a luciferase, a modified luciferase, a fluorescent protein, a natural protein, or a synthetic protein.

Further, the carrier protein may also be a mutated carrier protein wherein the mutation within the carrier protein results in an inhibition of enzymatic activity of the carrier protein such that the carrier protein does not express any detectable activity when the promoter region becomes active. The carrier protein with such a mutation can be fused to the ED enzyme fragment operably coupled to a promoter region, wherein the ED fragment, if expressed, combines with EA enzyme fragment to form ED-EA enzyme complex with enzymatic activity and measuring the enzyme activity to assess the activity of the promoter region of interest and hence activity of a transcription factor. Accordingly, the present invention discloses a use of a carrier protein with an enzymatic activity which is not same as the β-galactosidase enzyme and further discloses a carrier protein mutated to render the enzymatic activity of the carrier protein inactive.

In many embodiments, a carrier protein does not possess any intrinsic enzymatic activity. The carrier protein further comprises a domain selected to affect the stability of the β-galactosidase fragment e.g. an enzyme donor (ED) fragment-carrier protein fusion wherein a domain is selected to increase the stability of the ED-carrier protein fusion as compared to ED-carrier protein fusion lacking the domain. Further a carrier protein may also comprise a domain selected to destabilize the ED-carrier protein fusion as compared to ED-carrier protein fusion lacking the domain.

As summarized above, the methods of the present disclosure further include detecting the level of the enzymatic activity to assess activity of the promoter region. According to some embodiments, detecting the level of the enzymatic activity includes providing a substrate for the ED-EA complexes, wherein a detectable signal is generated upon hydrolysis of the substrate by the ED-EA complexes. In certain embodiments, the detectable signal is a chemiluminescent signal.

Aspects of the method include the use of a reduced-affinity enzyme complementation reporter system such as a β-galactosidase enzyme fragment complementation (EFC) reporter system. By “reduced-affinity” enzyme complementation reporter system is meant a system that is made up of two or more fragments of an enzyme (i.e., reporter subunits) that by themselves lack any of the detectable activity (which may be directly or indirectly detectable) that is observed in their parent enzyme but when brought sufficiently close together, e.g., through random interaction or a binding member mediated interaction, give rise to a detectable amount of the activity of the parent enzyme. An aspect of the reduced affinity enzyme complementation reporter systems of the invention is that at least one of the reporter subunits employed in the system is a variant of a corresponding domain in its wild-type parent enzyme such that its interaction with the other subunits of the system is reversible under assay conditions, absent an interaction mediated by binding moieties of interest. In this system a small fragment of β-galactosidase and a larger fragment of 1-galactosidase are employed, where the two fragments have a low affinity for each other. The small fragment of 1-galactosidase, enzyme donor (“ED”) may have the naturally occurring sequence or a mutated sequence. According to some embodiments, the ED is a β-galactosidase donor fragment. A variety of 1-galactosidase fragment EDs may be employed. In certain embodiments, when the ED is a β-galactosidase donor fragment, the ED comprises an amino acid sequence set forth in Table 3 below, or a variant thereof (e.g., a variant thereof having 10 or fewer, 8 or fewer, 6 or fewer, 5 or fewer, 4 or fewer, 3 or fewer, 2 or fewer, or 1 conservative amino acid substitution relative to an amino acid sequence set forth in Table 3) that complexes with the EA to form an enzyme having enzymatic activity. The amino acid sequences of example β-galactosidase donor fragment EDs are provided in Table 3 below.

The activity of β-galactosidase or the ED-EA complex forming an active β-galactosidase enzyme complex with enzyme activity may be detected using a chemiluminescence assay. For example, cells containing β-gal fusions are lysed in a mixture of buffers containing Galacton Plus substrate from a Galactolight Plus assay kit (Tropix, Bedford Mass.). Bronstein et al, J. Biolumin. Chemilumin., 4:99-111 (1989). After addition of Light Emission Accelerator solution, luminescence is measured in a luminometer or a scintillation counter. In many embodiments, the detection method for β-galactosidase enzyme activity also includes lysing the cell and detecting the enzyme activity of ED-EA enzyme fragment using any β-galactosidase substrate capable of yielding a detectable product such as direct chromogenic, fluorogenic, or chemiluminescent substrates or substrates of a coupled-assay with a bioluminescent readout.

TABLE 3
Example β-galactosidase donor fragment ED amino
acid sequences

| | |
|---|---|
| β-galactosidase | NSLAVVLQRRDWENPGVTQLNRLAAHPPFASW |
| donor fragment | RNSEEARTDR |
| ED | |
| (SEQ ID NO: 30) | |
| β-galactosidase | MGVITDSLAVVLQRRDWENPGVTQLNRLAAHP |
| donor fragment | PFASWRNSEEARTDRPSQQL |
| ED | |
| (SEQ ID NO: 31) | |
| β-galactosidase | MGVITDSLAVVLQRRDWENPGVTQLNRLAAHP |
| donor fragment | PFASYRNSEEARTDRPSQQL |
| ED | |
| (SEQ ID NO: 32) | |

As used herein, a “conservative substitution” is one in which an amino acid is substituted for another amino acid that has similar properties, such that one skilled in the art of peptide/protein chemistry would expect the secondary structure and hydropathic nature of the peptide/protein, or domain thereof, to be substantially unchanged. Modifications may be made in the structure of the polynucleotides and polypeptides contemplated in particular embodiments, and still obtain a functional molecule that encodes a variant or derivative polypeptide with desirable characteristics, e.g., the ability to complex with the EA to form an enzyme having glycoside hydrolase activity. When it is desired to alter the amino acid sequence of an ED, EA, or domain thereof to create an equivalent, or even an improved, variant ED or EA, one skilled in the art, for example, can change one or more of the codons of the encoding DNA sequence.

By “EA” is meant an enzyme acceptor fragment for use in an enzyme fragment complementation assay. In certain embodiments, the ED is a β-galactosidase donor fragment and the EA is a β-galactosidase acceptor fragment. By way of example, the ED may be an ED comprising an amino acid sequence set forth in Table 3 (or a variant thereof that complexes with the EA to form an enzyme having glycoside hydrolase activity), and the EA is a commercially available EA that complexes with the ED to form an enzyme having glycoside hydrolase activity. According to some embodiments, such an EA is provided in the PathHunter® ProLabel®/ProLink™ Detection Kit available from Eurofins DiscoverX, Corporation.

The methods of the present disclosure include contacting the ED, if expressed, with an EA to form ED-EA complexes having enzymatic activity. In some embodiments, the cell is intact when the ED is contacted with the EA. For example, the ED may be contacted with the EA when the cell is alive, when the cell is fixed, etc. When the cell is intact when the ED is contacted with the EA, the EA is generally a cell-permeable enzyme fragment such that the EA may cross the cell membrane to contact the ED expressed in the cell. When a β-galactosidase-based EFC system is employed, a range of methods are available to measure the enzyme activity of β-galactosidase which include live cell flow cytometry and histochemical staining with the chromogenic substrate 5-bromo-4-chloro-3-indolyl β-D-galactopyranoside (X-Gal). See e.g., Nolan et al., Proc. Natl. Acad. Sci., USA, 85: 2603-2607 (1988); and Lojda, Z., Enzyme Histochemistry: A laboratory Manual, Springer, Berlin (1979). Vital substrates for β-gal, which can be used in living cells, are also encompassed by the presently disclosed methods and materials. For example, a fluorogenic substrate, resorufin β-galactosidase bis-aminopropyl polyethylene glycol 1900 (RGPEG) has been described. Minden (1996) BioTechniques 20(1): 122-129. This compound can be delivered to cells by microinjection, electroporation or a variety of bulk-loading techniques. Once inside a cell, the substrate is unable to escape through the plasma membrane or by gap junctions. Another vital substrate that can be used in the practice of the presently disclosed methods and materials is fluorescein di-β-D-galactopyranoside (FDG), which is especially well-suited for analyses by fluorescence-activated cell sorting (FACS) and flow cytometry. Nolan et al., Proc. Natl. Acad. Sci, USA, 85:2603-2607 (1988) and Rotman et al. (1963) Proc. Natl. Acad. Sci, USA 50:1-6.

In some embodiments, the methods further include lysing the cell, and contacting the ED with the EA includes combining the cell lysate with the EA. Any suitable lysis agent (e.g., lysis buffer) may be used to lyse the cells. Non-limiting examples of lysis buffers include NP-40 lysis buffer, RIPA (RadioImmuno Precipitation Assay) lysis buffer, SDS (sodium dodecyl sulfate) lysis buffer, ACK (Ammonium-Chloride-Potassium) lysing buffer, and the like. The lysis buffer may include buffering salts (e.g., Tris-HCl) and/or ionic salts (e.g., NaCl) to regulate the pH and osmolarity of the lysate. Detergents (such as Triton X-100 or SDS) may be added to disrupt the cell membrane structures. The lysis buffer may include additional useful components such as protease inhibitors, etc. When the methods include lysing the cell and a β-galactosidase-based EFC system is employed, active reconstituted β-galactosidase may be detected using a chemiluminescence assay. For example, cells containing reconstituted β-galactosidase (via EFC) may be lysed (with or without contacting with a crosslinking agent) in a mixture of buffers containing Galacton Plus substrate from a Galactolight Plus assay kit (Tropix, Bedford Mass.). Bronstein et al, J. Biolumin. Chemilumin., 4:99-111 (1989). After addition of Light Emission Accelerator solution, luminescence is measured in a luminometer or a scintillation counter. In some embodiments, when the methods include lysing the cell and a β-galactosidase-based EFC system is employed, the PathHunter® ProLabel®/ProLink™ or KILR® Detection Kits available from Eurofins DiscoverX Corporation may be employed to detect the enzymatic activity by chemiluminescence.

Also provided by the present disclosure are cells. The cells find use in practicing the methods of the present disclosure. A cell of the present disclosure may include any of the nucleic acids of the present disclosure that include a region that encodes an enzyme donor (ED) operably coupled to a promoter region, including any of the nucleic acids described above in the present Methods section and the Experimental section below, which are incorporated but not reiterated herein for purposes of brevity. In some embodiments, the cell has any characteristics (e.g., may be any of the cell types, etc.) of the cells described above in the present Methods section and the Experimental section below, which are incorporated but not reiterated herein for purposes of brevity.

### Compositions

As summarized above, the present disclosure also provides compositions. In certain embodiments, the compositions find use, e.g., in practicing the methods of the present disclosure. According to some embodiments, a composition of the present disclosure includes any of the nucleic acids and/or any of the cells of the present disclosure, including any of the nucleic acids and/or cells described in the Methods section above and Experimental section below, which are incorporated but not reiterated herein for purposes of brevity.

A composition of the present disclosure may include any of the nucleic acids and/or any of the cells of the present disclosure, present in a liquid medium. The liquid medium may be an aqueous liquid medium, such as water, a buffered solution, a cell culture medium (e.g., DMEM, RPMI, MEM, IMDM, DMEM/F-12, or the like), or the like. One or more additives such as an antibiotic, a salt (e.g., NaCl, MgCl₂, KCl, MgSO₄), a buffering agent (a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

In certain embodiments, provided is a composition that includes any of the cells of the present disclosure, present in a buffered liquid medium. According to some embodiments, the liquid medium is a cell culture medium, e.g., DMEM, RPMI, MEM, IMDM, DMEM/F-12, or the like. In certain embodiments, provided is a composition that includes any of the nucleic acids of the present disclosure, present either in a lyophilized form, or present in a buffered liquid medium.

The compositions of the present disclosure may be present in any suitable container, such as a tube, vial, ampule, one or more wells of a plate, e.g., 4-, 6-, 8-, 12-, 24-, 48-, 96-, 384-, 1536-well tissue culture plate, or the like.

### Kits

Aspects of the present disclosure further include kits. In certain embodiments, the kits find use, e.g., in practicing the methods of the present disclosure. According to some embodiments, a kit of the present disclosure includes any of the nucleic acids, cells and/or compositions of the present disclosure, including any of the any of the nucleic acids, cells and/or compositions described in the Methods and Compositions sections above and the Experimental section below, which are incorporated but not reiterated herein for purposes of brevity.

In certain embodiments, provided is a kit that comprise a cell including a nucleic acid comprising a region that encodes an enzyme donor (ED) operably coupled to a promoter region, and instructions for using the cell to perform any of the methods of the present disclosure. For example, the kits may comprise instructions for assessing activity of the promoter region of the nucleic acid. The kits may comprise instructions (and any reagents useful) for any of the culturing, contacting, detecting, etc. steps described in the Methods section above and the and the Experimental section below.

In many embodiments, provided is a kit that includes a cell comprising a nucleic acid comprising a region that encodes a carrier protein fused to an ED operably coupled to a promoter region, and instructions for using the cell to perform any of the methods of the present disclosure.

The kits of the present disclosure may further include instructions for contacting the cell with an agent (e.g., a control agent, a test agent, and/or the like) during the culturing, and assessing the activity level of the promoter region in response to contacting the cell with the agent (e.g., a small molecule, protein (e.g., a cell surface protein), nucleic acid, etc.) based on the detected level of the enzymatic activity. Such kits may further include instructions for contacting the cell with a control agonist that activates the cell signaling pathway of interest. Such a kit may further include the control agonist. The activity level of the promoter region may be used as the basis for assessing the effect of the agent on a transcription factor of interest and/or a cell signaling pathway of interest. The instructions may include instructions for making such an assessment.

According to some embodiments, the ED encoded by the nucleic acid present in the cell of the kits is a β-galactosidase donor fragment ED. For example, the ED may include an amino acid sequence of an example β-galactosidase donor fragment ED set forth in Table 3, or a variant thereof capable of complexing with an EA to form an enzyme having glycoside hydrolase activity. In certain embodiments, a kit of the present disclosure includes the EA. For example, when the nucleic acid of the cell encodes a β-galactosidase donor fragment ED, the EA included in the kit may be a β-galactosidase acceptor fragment EA selected such that the ED-EA pair produces a functional enzyme having glycoside hydrolase activity via EFC.

According to some embodiments, a kit of the present disclosure further includes instructions for lysing the cell prior to contacting the ED with the EA. In certain embodiments, a kit of the present disclosure includes a lysing agent. Non-limiting examples of lysis buffers that may be included in a kit of the present disclosure include NP-40 lysis buffer, RIPA (RadioImmuno Precipitation Assay) lysis buffer, SDS (sodium dodecyl sulfate) lysis buffer, ACK (Ammonium-Chloride-Potassium) lysing buffer, and the like. In other embodiments, a kit of the present disclosure further includes instructions for contacting the ED with the EA when the cell is intact. Such a kit may include instructions for contacting the ED with the EA in a live cell (and detecting by flow cytometry, etc.), in a fixed intact cell, etc.

Components of the kits may be present in separate containers, or multiple components may be present in a single container. Suitable containers include individual tubes (e.g., vials), ampoules, wells of one or more plates, or the like.

The instructions provided with a kit may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

The following examples are offered by way of illustration and not by way of limitation.

### EXPERIMENTAL

### Example 1—NFAT EFC Reporter Construct

NFAT EFC Reporter construct includes a promoter with 4 tandemly repeated (4×) NFAT transcription factor binding response elements, each having the sequence: GGAGGAAAAACTGTTTCATACAGAAGGCGT (SEQ ID NO:5). PEST is a protein destabilizing sequence (a peptide sequence that is rich in proline (P), glutamic acid (E), serine (S), and threonine (T)) which enhances the proteasome-mediated turnover of the reporter-enhanced ProLabel (ePL) protein. ePL protein is an inactive ˜45 amino acid fragment of β-Galactosidase. Reducing the lifetime of the carrier protein has been shown previously to shorten the response time required for the assay and possibly increase the sensitivity to ligand concentration (Fan and Wood, Assay Drug Dev Technol. 2007 February; 5(1):127-36).

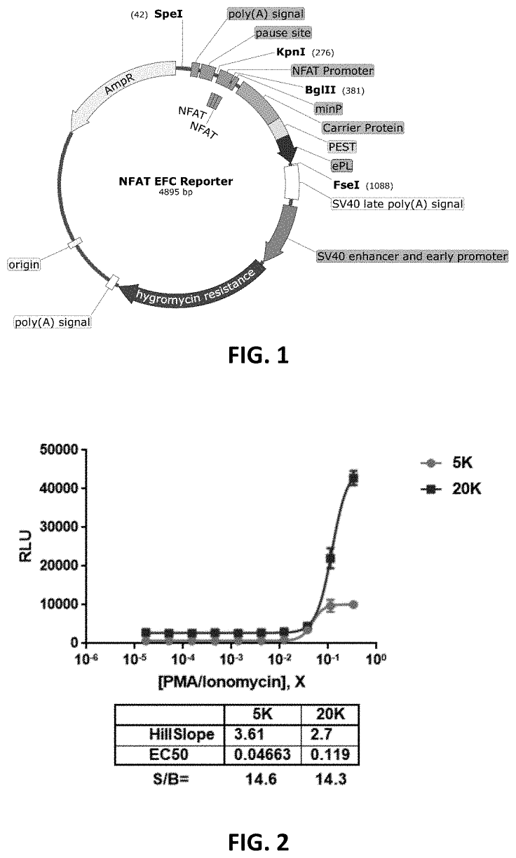

FIG. 1 shows the plasmid map for the NFAT EFC reporter construct used for EFC-based assays of NFAT transcription factor activation and activation of signaling pathways impinging on NFAT. Different promoter elements (e.g. 4× response NFAT elements in this example), different carrier proteins and different destabilizing motifs (e.g. PEST in this example) may be used to target and tune these constructs for different applications.

FIG. 2 shows the dose response curve (DRC) of U2OS NFAT EFC Reporter cells to a “cell stimulation” cocktail of phorbol 12-myristate 13-acetate (PMA) and ionomycin (data is expressed as fold 1× where 1× cocktail is 81 nM PMA/1.34 μM Ionomycin cell stimulation). Cells were stimulated for 20h followed by detection by PathHunter® FLASH detection reagent (+Enzyme Acceptor (EA)) with cell lysis for 1h. 5K and 10K refers to the number of cells plated in each well of a 384 well plate. S/B is signal (top) over background (bottom) of the assay response curve.

A “cell stimulation” cocktail of phorbol 12-myristate 13-acetate (PMA) and ionomycin is known to activate “NFAT signaling pathways” and the NFAT transcription factor. Stimulation of U2OS cells stably transfected with NFAT EFC Reporter construct (see FIG. 1) using different dilutions of PMA/ionomycin cocktail (where 1λ=81 nM PMA/1.34 μM Ionomycin) for 20h revealed a dose dependent stimulation of the NFAT EFC reporter as evidenced by EFC after addition of +EA. The potency of the cocktail was shown to be 0.047× (or 3.8 nMPMA/63 nM ionomycin) using 5,000 cells. These data demonstrate that U2OS NFAT EFC Reporter cells are suitable to develop assays for activation or inhibition of “NFAT signaling pathways” and NFAT transcription factor. Inhibition could be detected as decreases in relative light units (RLUs) with test compounds in the presence of stimulating doses of cell stimulation cocktail or other activating ligands.

### Example 2—NFkB EFC Reporter Construct

NF-kB EFC Reporter Plasmid includes a promoter that has 5 tandemly repeated NF-κB transcription factor binding response elements, where of 3 GGGAATTTCC (SEQ ID NO:6) sequences are interspersed by 2 alternating GGGGACTTTCC (SEQ ID NO:6) sequences.

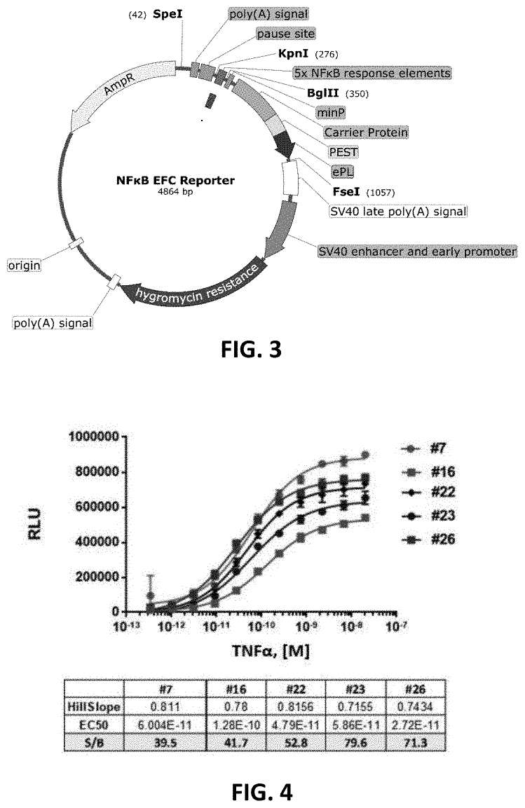

FIG. 3 shows the plasmid map for the NF-κB EFC Reporter Plasmid used for EFC-based assays of NF-κB transcription factor activation and activation of NF-κB signaling pathways. Different carrier proteins and different destabilizing motifs (e.g. PEST in this example) may be used to tune these constructs for different applications.

FIG. 4. Response to cytokine TNFα of single-cell clones of U20S NF-κB EFC Reporter cells. 2500 cells/well from 5 individual single-cell clones of a U2OS cells stably expressing NF-κB EFC Reporter construct were plated in a 384 well plate. Cells were stimulated with the indicated concentrations of TNFα for 6h followed by detection by PathHunter® FLASH detection reagent (+5×EA) with cell lysis.

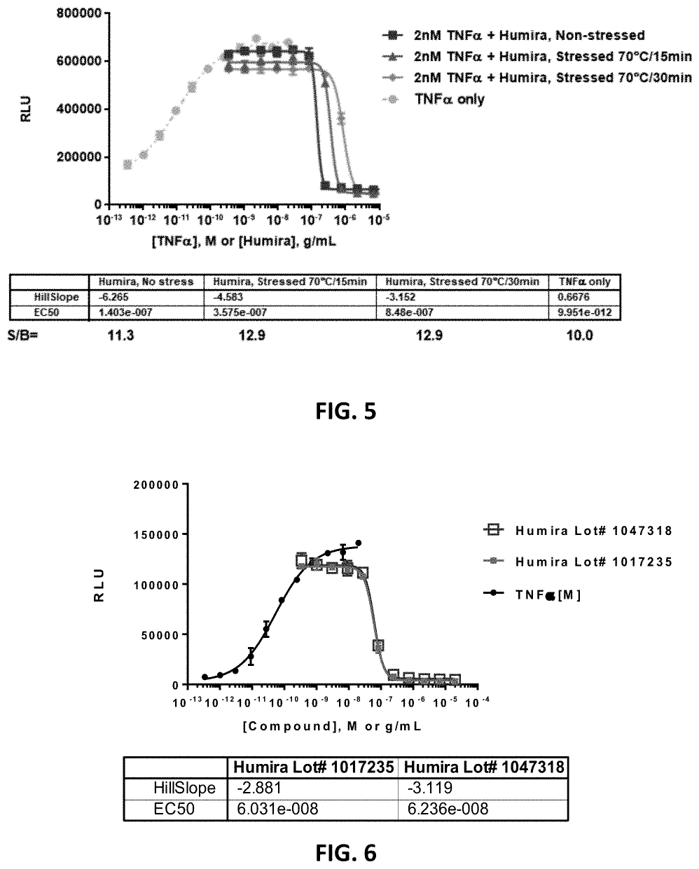

TNFα is known to stimulate the “NF-κB signaling pathways” and the NF-κB transcription factor. Stimulation of U2OS cells stably transfected with NF-κB EFC Reporter construct using different dilutions of TNFα for 6h reveal a dose dependent stimulation of the NF-κB EFC reporter as evidenced by EFC after addition of +EA (FIG. 4). The potency of TNFα was shown to be 0.03-0.12 nM for the different clones. These data demonstrate that U2OS NF-κB EFC Reporter cells are able to respond sensitively to cytokine TNFα indicating the possibility for development of assays for activation or inhibition of NF-κB signaling pathways and NF-κB transcription factor. Assays for inhibition could be developed by looking for decreases in RLUs with test compounds in the presence of stimulating doses of TNFα is or other activating ligands (see FIG. 5).

FIG. 5. Screening of inhibitors using U2OS NFkB EFC reporter cell-based assay. U2OS NFkB EFC reporter cells were treated with 2 nM TNFα (agonist) with or without various concentrations (and 3 lots) of anti-TNFα inhibitor adalimumab. In this example, the 3 lots of Humira tested were all derived from lot #1047318 run through a forced degradation protocol to simulate decreased specific activity or loss of activity where samples were non-stressed (e.g. the control), stressed 70° C. for 15 min and stressed 70° C. for 30 min.

U2OS NF-κB EFC Reporter cells can also be used to measure inhibition and study inhibitors of NF-κB signaling. U2OS NF-κB EFC reporter assay was used to demonstrate that adalimumab (Humira®) can inhibit TNFα-stimulated NF-κB signaling (FIG. 5). The dose-dependence of this inhibitory response allowed measurement of the potency of different lots of adalimumab. FIG. 5 Humira® shows changes in the inhibitory potency of Humira® samples subjected to different amounts of forced degradation by heating. Note that forced degradation is a method used in development of assay for biologics (protein therapeutics) to simulate lots with different potency, e.g. for QC/lot release assays, or loss of potency from various causes. The U2OS NF-κB EFC Reporter assay detected a graded loss of potency (right shift) of Humira® samples subjected to forced degradation for 0, 15 or 30 min at 70° C. The stability of Humira® over time can also be studied using this assay. It can be seen that two lots of Humira® stored at 4° C. (both commercially procured and, thus, whose potency would have been essentially identical at the time of release), one non-expired (#1047318) and one over 14 months past expiration (#1017235), were shown to possess the same NF-κB pathway inhibitory potency in the TNFα-stimulated U2OS NF-κB EFC Reporter assay (FIG. 6).

FIG. 6. The NFkB EFC Reporter Assay revealed that two Humira® lots tested had identical TNFα inhibitory activity and potency, despite lot #1017235 having expired over 14 months prior (Humira® lot #1047318 was non-expired). The stability of expired lot #1017235 was also equivalent to that of lot #1047318 in the forced degradation experiment shown in FIG. 5.

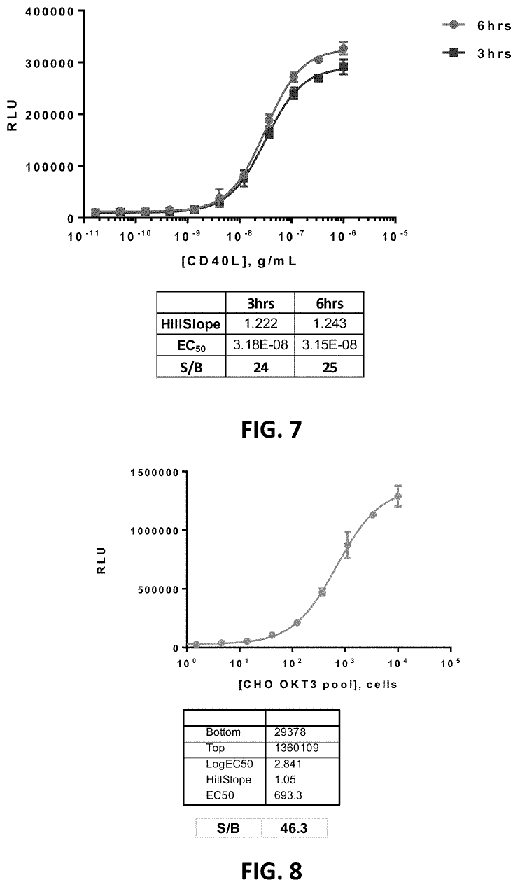

FIG. 7. Endogenous CD40 receptor in NFkB EFC Reporter Assay Cells responded robustly to CD40 ligand (CD40L) after either 3h or 6h incubation. These same cells also endogenously express TNF receptor and respond to soluble TNFα (as seen in FIGS. 4-6).

Often multiple ligands acting through different cellular receptors will stimulate the same signaling pathway allowing the same EFC reporter assay to be used to study the function and inhibition of function for these multiple ligands. For example, in FIGS. 4-6 stimulation and inhibition of TNFα-stimulated NFkB signaling and gene expression was studied. Using the same assay, the study in FIG. 7 shows that CD40L acting through the CD40 receptor also stimulates this same transcription factor and gene expression pathway.

### Example 3—NFAT EFC Reporter Cell Stimulation in Co-Culture Assay

Many ligands whose activity and inhibition are of current research interest are soluble extracellular ligands such as TNF, CD40L or soluble intracellular ligands such as PMA and ionomycin. However, these same cell-based EFC reporter assays can also be used to study cell-associated ligands presented to the assay cells on the surface of another cell (e.g. cell-cell or intercellular interaction). This other ligand presenting cell can be a heterologous or autologous cell with respect to the assay cells. FIG. 8 shows that OKT3 ligand presented on the surface of CHO-K1 cells can activate NFAT-mediated gene expression in a Jurkat NFAT EFC reporter cell pool.

FIG. 8. Jurkat NFAT EFC reporter cells respond to OKT3 ligand expressed and presented on the surface of CHO-K1 cells in a co-culture assay. OKT3 is comprised of a CD5 leader peptide fused to single chain antibody fragment of murine anti human T-cell receptor CD3 subunit fused to leaderless human CD14; accession number HM208750.1. Addition of CHO OKT3 cells stimulated carrier protein expression of NFAT EFC reporter in a graded manner with an EC50 of stimulating OKT3 cells of ˜700 cells.

### Example 4—IL2-Promoter-EFC Reporter Construct

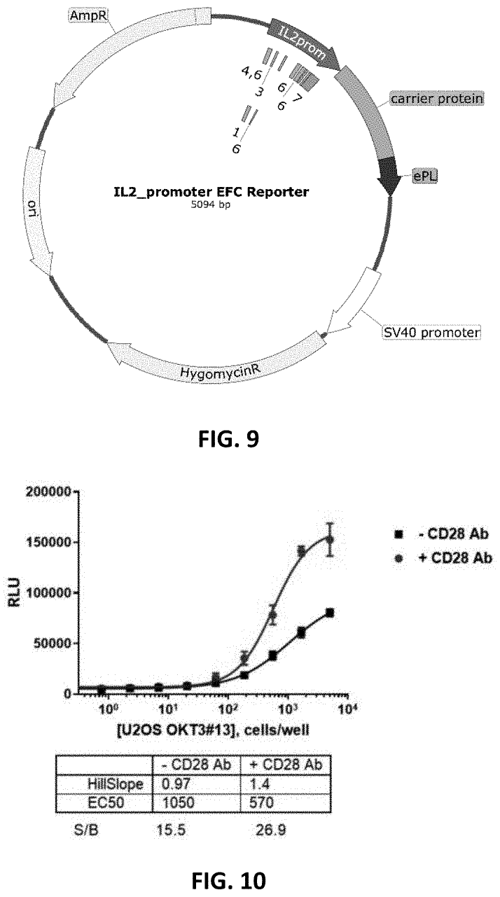

FIG. 9. Plasmid map for IL2-Promoter-EFC Reporter Plasmid. In this reporter construct, the complete endogenous IL2 gene promoter (“IL2prom”), which contains multiple specific transcription factor binding sites is fused upstream of and drives expression of carrier protein-ePL which is fused to a carrier protein. Contained within the IL2 promoter are binding sites for NFAT (1), NFkB (2), OCT (3), ARRE-2 (4) and NFAT/API (6) transcription factors and the IL2 minimal promoter (7). The DNA sequence of the IL2 promoter used is gtacCTTTTCTGAGTTACTTTTGTATCCCCACCCCCTTAAAGAAAGGAGGAAAAACTGT TTCATACAGAAGGCGTTAATTGCATGAATTAGAGCTATCACCTAAGTGTGGGCTAAT GTAACAAAGAGGGATTTCACCTACATCCATTCAGTCAGTCTTTGGGGGTTTAAAGAA ATTCCAAAGAGTCATCAGAAGAGGAAAAATGAAGGTAATGTTTTTTCAGACAGGTAA AGTCTTTGAAAATATGTGTAATATGTAAAACATTTTGACACCCCCATAATATTTTTCC AGAATTAACAGTATAAATTGCATCTCTTGTTCAAGAGTTCCCTATCACTCTCTTTAAT CACTACTCACAGTAACCTCAACTCCTGCCAgctag (SEQ ID NO: 11) and is comprised of 8 different transcription factor binding sites and an IL-2 core promoter as described by Weaver et. al. (Molecular Immunology, 6 Mar. 2007, 44(11):2813-2819). The following 8 DNA elements ACCCCCTTAAAGAAAGGAGGAA (SEQ ID NO: 12), GGAGGAAAAACTGTTTCATACAGAAGGCGT (SEQ ID NO:13), AATTGCATGAA (SEQ ID NO:14), GGGATTTCACC (SEQ ID NO:15), ATGAAGGTAATGTTTTTTCAG (SEQ ID NO:16), GTCTTTGAAAATATGTGTAAT (SEQ ID NO:17), AAACATTTTG (SEQ ID NO:18) and TAATATTTTT (SEQ ID NO:19) respond to transcription factors NFAT & API, NFAT, OCT, NFκB, NFAT & API, NFAT & API, OCT, NFAT, respectively, while the IL-2 core promoter is CAGAATTAACAGTATAAATTGCATCTCTTGTTCAAGAGTTCCCTATCACTCT (SEQ ID NO:20) with the TATA box is underlined.

FIG. 10. The Jurkat IL2-promoter EFC reporter cell line can detect the stimulation of multiple distinct response elements through activation of different signaling pathways. 5K Jurkat IL2 promoter reporter cells were plated in wells of 384 well plate and stimulated for 16h with either OKT3 cells (squares) or OKT3 cells+CD28 antibody (circles) at 37′C prior to adding FLASH detection reagent (+5×EA) with cell lysis (and reading EFC luminescence). The EC₅₀ is indicated as the number of OKT3-presenting CHO-K1 cells per well.

OKT3-bearing CHO-K1 cells stimulate Jurkat cell IL2-promoter reporter expression which is believed to occur primarily through activation of NFAT response elements (FIG. 10, squares). Anti-CD28 antibody, which is believed to act primarily through NFkB response elements, augments the OKT3 stimulation of the IL2 promoter (FIG. 10, circles) as evidenced by a ˜2-fold increase in the signal and ˜2-fold decrease in EC₅₀ for OKT3 cells alone. Comparing the response of OKT3+anti-CD28 to that of OKT3 alone demonstrates the additive (or synergistic) induction of IL2 promoter EFC reporter by stimulating multiple response elements and signaling pathways. Thus, this more complex physiologic promoter can be used to study more complex and integrative regulation of the native IL2 promoter more similar to what is seen for regulation of IL2 expression and secretion in vivo.

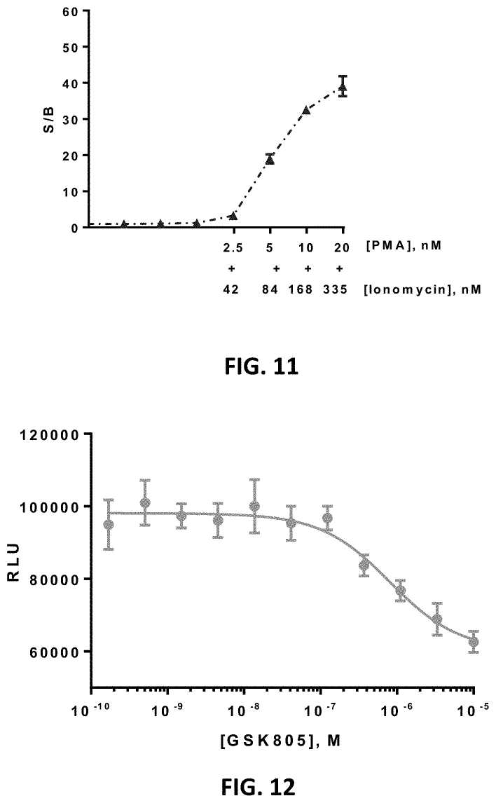

FIG. 11. Response of an IL2-promoter EFC reporter construct, a complex, native promoter with multiple different response elements, to intracellular mimics of two different signaling pathways, Phorbol ester and ionomycin. 5K cells were plated in wells of 384 well plate and stimulated for 16h prior to adding FLASH detection reagent (+5×EA) with cell lysis (and reading luminescence). S/B is calculated by taking the RLU (with PMA/ionomycin)/RLU (without PMA/ionomycin).

Ionomycin and phorbol ester PMA via stimulation of NFAT and AP-1 response elements, respectively, increase the activation of distinct elements in the IL2-promoter EFC reporter construct (FIG. 11). Like the example shown in FIG. 10, this shows that multiple inputs can be used to study more complex and integrative regulation of the native IL2 promoter more similar to what is seen for regulation of IL2 expression and secretion in vivo.

### Example 5—Promoter-EFC Reporter Construct for Assaying for an Antagonist

FIG. 12. Activity of RORγT transcription factor is decreased by inverse agonist GSK805 in U2OS cells expressing RORγT transcription factor and RORγT EFC reporter plasmid. Cells were treated with GSK805 (Tocris) for 18h, then expression of carrier protein-ePL was detected using EFC by addition of EA plus PathHunter® FLASH detection reagent.

Specific EFC reporter constructs can be used to study the activity of inverse agonists (an inverse agonist is a ligands that bind directly to the transcription factor or receptor and decrease its activity below basal levels) on transfected transcription factors. In this case, inverse agonist GSK805 was shown to decrease the activity of RORγT transcription factor for stimulation of the RORγT EFC reporter in U20S cells (FIG. 12).

### Example 6—EFC-Based NFκB Transcriptional Reporter Assay Exhibits Better Sensitivity than the Luciferase System

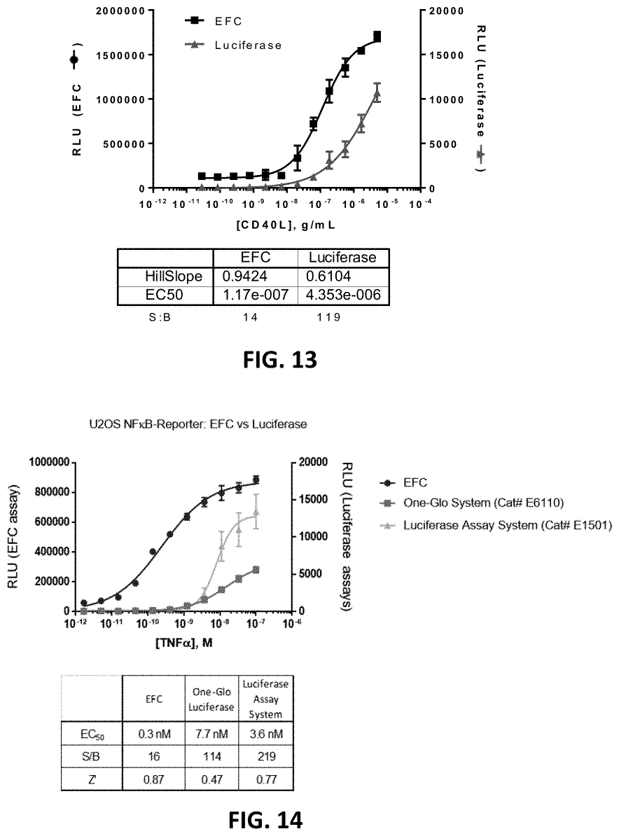

FIG. 13. The EFC-based NFκB transcriptional reporter assay exhibits better sensitivity to CD40L/CD40 receptor than the Luciferase system. U2OS cells stably transfected with a plasmid encoding an NFκB transcriptional response element driving expression of either the EFC reporter (left panel) or (Firefly) Luciferase-PEST were stimulated with a range of concentrations of CD40L for 6h followed by cell lysis, addition of excess EA, incubation for 1h and determination of luminescence (RLU). The luminescence detected indicates the extent of induction of the respective carrier protein by CD40L.

The EFC-based NFκB transcriptional reporter assay was over 35 times more sensitive to CD40L than the luciferase-based assay. Note that the reporter plasmids used to make the stable cell lines had identical elements with the only significant difference being the identity of the carrier protein. Specifically, both plasmids had the same promoter elements and both had the same protein destabilizing element (PEST sequence). Thus, for CD40L, reporter assay detection EFC was significantly more sensitive than detecting luciferase activity.

FIG. 14. The EFC-based NFκB transcriptional reporter assay exhibits better sensitivity than the Luciferase system. U2OS cells stably transfected with a plasmid encoding an NFκB transcriptional response element driving expression of either the EFC reporter (left panel) or (Firefly) Luciferase-PEST were stimulated with a range of concentrations of TNFα for 18h followed by cell lysis, addition of excess EA, incubation for 1h and determination of luminescence (RLU). The luminescence detected indicates the extent of induction of the respective carrier protein by TNFα.

FIG. 14 shows that the EFC-based NFκB transcriptional reporter assay exhibits better sensitivity to TNF receptor ligand TNFα than the Luciferase system. The ligand TNFα is 12-15 more potent in the EFC assay (left panel) than the Luciferase assays (right panel) which can be advantageous in detection of weak response to a candidate agent in a screening assay, for example.

Increased sensitivity for ligand stimulation by EFC-based reporter assay was observed for both CD40L and TNFα. This increased sensitivity is beneficial and important because it allows using the EFC reporter assay to study less potent compounds as might be found earlier in the affinity maturation of a chemical inhibitor (such as in the hit discovery or early in hit-to-lead optimization phases of drug discovery).

### Example 7—Cell Line for NFκB Pathway Reporter Assay

A reporter cell line was engineered to express an Enzyme Donor (ED) tagged carrier protein controlled by a pathway-inducible transcriptional response element. Pathway activation results in induced expressions of the ED-tagged protein. Addition of exogenous Enzyme Acceptor (EA), and buffer, lyses the cell and forces complementation of the ED and EA enzyme fragments. This results in the formation of a functional enzyme that hydrolyzes substrate to generate a chemiluminescent signal.

In this example, the cell line is an NFκB (nuclear factor NF-kappa-B p100 subunit) pathway reporter cell line. The cells are U2OS cells that include a nucleic acid encoding a β-galactosidase donor fragment (ED) having the amino acid sequence NSLAVVLQRRDWENPGVTQLNRLAAHPPFASWRNSEEARTDR (SEQ ID NO:30) operably coupled to a promoter region comprising an NFκB response element.

Cells were plated in a 96-well plate and incubated at 37° C. and 5% CO₂ to allow the cells to attach and grow. Cells were then stimulated with a control agonist (here, CD40L), using the assay conditions described below. Following stimulation, signal was detected using the PathHunter® ProLabel®/ProLink™ Detection Kit (Eurofins DiscoverX Corporation) according to the recommended protocol.

### Assay Conditions

| | |
|---|---|
| Cell Number/Well | 5000 |
| Cell Seeding Time hours | 24 |
| Control Agonist | CD40L |
| Ligand Incubation Time (minutes) | 360 |
| Ligand Incubation Temperature (° C.) | 37 |

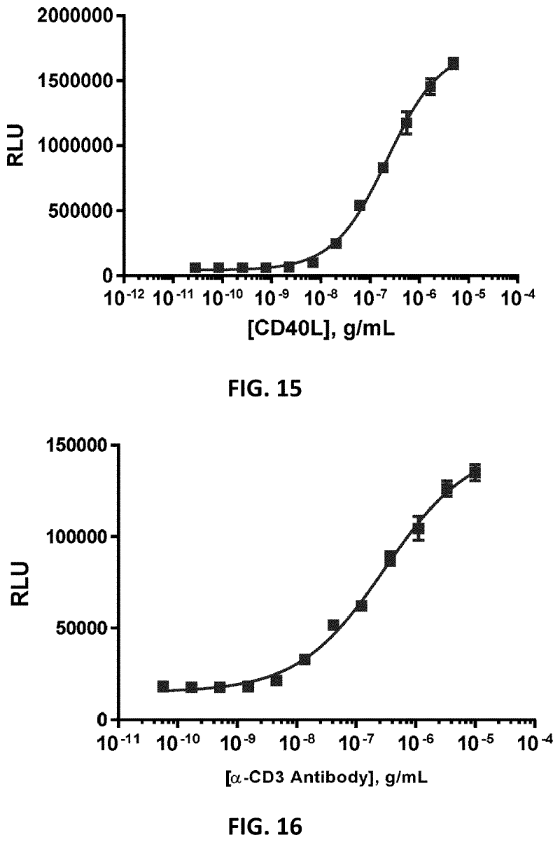

Results are shown in FIG. 15. This reporter cell line exhibited an EC₅₀ for control agonist stimulation of 225.4 ng/mL and a signal:background ratio at agonist E_{MAX} of 26.3.

The cell line was confirmed to be stable through 10 passages with no significant drop in assay window or change in EC₅₀.

### Example 8—Cell Line for NFAT Pathway Reporter Assay

A reporter cell line was engineered to express an Enzyme Donor (ED) tagged carrier protein controlled by a pathway-inducible transcriptional response element. Pathway activation results in induced expressions of the ED-tagged protein. Addition of exogenous Enzyme Acceptor (EA), and buffer, lyses the cell and forces complementation of the ED and EA enzyme fragments. This results in the formation of a functional enzyme that hydrolyzes substrate to generate a chemiluminescent signal.

In this example, the cell line is an NFAT (nuclear factor of activated T-cells) pathway reporter cell line. The cells are Jurkat cells that include a nucleic acid encoding a β-galactosidase donor fragment (ED) having the amino acid sequence NSLAVVLQRRDWENPGVTQLNRLAAHPPFASWRNSEEARTDR (SEQ ID NO:30) operably coupled to a promoter region comprising an NFAT response element.

Cells were plated in a 96-well plate and incubated at 37° C. and 5% CO₂ to allow the cells to attach and grow. Cells were then stimulated with a control agonist (here, anti-CD3 antibody), using the assay conditions described below. Following stimulation, signal was detected using the PathHunter® ProLabel®/ProLink™ Detection Kit (Eurofins DiscoverX Corporation) according to the recommended protocol.

### Assay Conditions

| | |
|---|---|
| Cell Number/Well | 20000 |
| Cell Seeding Time (hours) | 24 |
| Control Agonist | anti-CD3 antibody |
| Ligand Incubation Time | Overnight |
| Ligand Incubation | 37 |
| Temperature (° C.) | |

Results are shown in FIG. 16. This reporter cell line exhibited an EC₅₀ for control agonist stimulation of 302.5 ng/mL and a signal:background ratio at agonist E_{MAX} of 7.6.

For this assay, the anti-CD3 antibody [OKT3] was pre-coated in the wells by plating 50 L of a 1:3 dilution series made in PBS and incubating the plate overnight at 4° C. Antibody was removed from the wells just prior to plating cells for the assay.

The cell line was confirmed to be stable through 10 passages with no significant drop in assay window or change in EC₅₀.

### Example 9—Cell Line for STAT3 Pathway Reporter Assay

A reporter cell line was engineered to express an Enzyme Donor (ED) tagged carrier protein controlled by a pathway-inducible transcriptional response element. Pathway activation results in induced expressions of the ED-tagged protein. Addition of exogenous Enzyme Acceptor (EA), and buffer, lyses the cell and forces complementation of the ED and EA enzyme fragments. This results in the formation of a functional enzyme that hydrolyzes substrate to generate a chemiluminescent signal.

In this example, the cell line is a STAT3 (signal transducer and activator of transcription 3) pathway reporter cell line. The cells are HepG2 cells that include a nucleic acid encoding a β-galactosidase donor fragment (ED) having the amino acid sequence NSLAVVLQRRDWENPGVTQLNRLAAHPPFASWRNSEEARTDR (SEQ ID NO:30) operably coupled to a promoter region comprising a STAT3 response element.

Cells were plated in a 96-well plate and incubated at 37° C. and 5% CO₂ to allow the cells to attach and grow. Cells were then stimulated with a control agonist (here, IL-6), using the assay conditions described below. Following stimulation, signal was detected using the PathHunter® ProLabel®/ProLink™ Detection Kit (Eurofins DiscoverX Corporation) according to the recommended protocol.

### Assay Conditions

| Cell Plating Reagent | CP5 |
|---|---|
| Cell Number/Well | 5000 |
| Plate Type | 96 Well |
| Cell Seeding Time (hours) | 4 |
| Control Agonist | IL-6 |
| Ligand Incubation Time | 16 h |
| Ligand Incubation Temperature (° C.) | 37 |

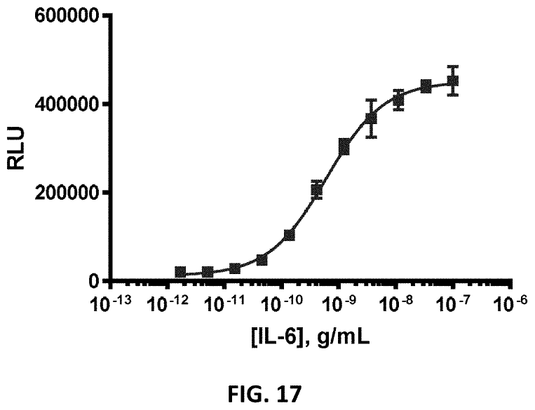

Results are shown in FIG. 17. This reporter cell line exhibited an EC₅₀ for control agonist stimulation of 0.601 ng/mL and a signal:background ratio at agonist E_{MAX} of 21.3.

The cell line was confirmed to be stable through 10 passages with no significant drop in assay window or change in EC₅₀.

Cell line for HepG2 STAT3 assay uses endogenous IL-6 receptor in HepG2 cells to detect IL-6 signaling.

### Example 10-Jurkat NFAT Pathway Reporter Assay

A reporter cell line was engineered to express an Enzyme Donor (ED) tagged carrier protein controlled by a NFAT pathway-inducible transcriptional response element. NFAT pathway activation results in activation of the NFAT transcription factor which binds to the NFAT pathway-inducible transcriptional response element and induces expressions of the ED-tagged carrier protein. Addition of exogenous Enzyme Acceptor (EA), and buffer, lyses the cell and forces complementation of the inactive ED and EA β-galactosidase enzyme fragments. This results in the formation of a functional β-galactosidase enzyme that hydrolyzes substrate to generate a chemiluminescent signal.

In this example, the cell line is an NFAT (nuclear factor of activated T-cells) reporter cell line. NFAT pathway activation results in activation of the NFAT transcription factor which binds to the NFAT pathway-inducible transcriptional response element and induces expressions of the ED-tagged carrier protein. The cells are Jurkat cells that include a nucleic acid encoding a β-galactosidase donor fragment (ED) having the amino acid sequence NSLAVVLQRRDWENPGVTQLNRLAAHPPFASWRNSEEARTDR (SEQ ID NO:30) operably coupled to a promoter region comprising a NFAT response element.

Cells were plated in a 96-well plate and incubated at 37° C. and 5% CO₂ to allow the cells to attach and grow. Cells were then stimulated with a control agonist (here, anti-CD-3 antibody), using the assay conditions described below. Following stimulation, signal was detected using the PathHunter® ProLabel®/ProLink™ Detection Kit (Eurofins DiscoverX Corporation) according to the recommended protocol.

### Assay Conditions

| | |
|---|---|
| Cell Number/Well | 20000 |
| Cell Seeding Time (hours) | N/A |
| Control Agonist | Anti-CD3 antibody |
| Ligand Incubation Time | Overnight |
| Ligand Incubation | 37 |
| Temperature (° C.) | |

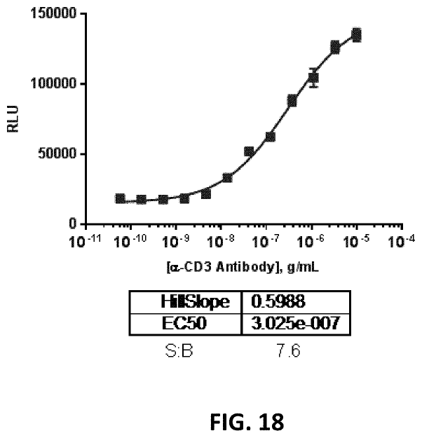

Results are shown in FIG. 18. This reporter cell line exhibited an EC₅s for control agonist stimulation of 3.025 ng/mL and a signal:background ratio at agonist E_{MAX} of 7.6.

For this assay, an activating T Cell Receptor (TCR) antibody, CD3 antibody, is pre-coated in the wells by plating 10 μg/ml and incubating the plate for 20 hrs. Antibody was removed from the wells just prior to plating cells for the assay.

The cell line was confirmed to be stable through 10 passages with no significant drop in assay window or change in EC₅₀.

### Example 11-Pathway Reporter Assay can be Further Modified to Generate Assays for Other Targets (Such as Ligands and Receptors)

A reporter cell line was engineered to express an Enzyme Donor (ED) tagged carrier protein controlled by a pathway-inducible transcriptional response element. Pathway activation results in induced expressions of the ED-tagged protein. Addition of exogenous Enzyme Acceptor (EA), and buffer, lyses the cell and forces complementation of the ED and EA enzyme fragments. This results in the formation of a functional enzyme that hydrolyzes substrate to generate a chemiluminescent signal.

In this example, the assay comprise of a co-culture of first cell line and a second cell line. The first cell line in the co-culture assay is the Jurkat PD1 (programmed cell death-1) pathway reporter cell line derived by expressing PD1 in the above-developed Jurkat NFAT Pathway Reporter cells. The cells into which PD1 was added are Jurkat cells that include a nucleic acid encoding a β-galactosidase donor fragment (ED) having the amino acid sequence NSLAVVLQRRDWENPGVTQLNRLAAHPPFASWRNSEEARTDR (SEQ ID NO:30) operably coupled to a promoter region comprising a NFAT Pathway response element. The second cell line in the PD1 pathway reporter co-culture assay is a U2OS cell line co-expressing PD-L1 (Programmed death ligand 1) and a TCR (T cell receptor) activator molecule. PD1 binding to its ligand PDL1 (Programmed death ligand 1) inhibits activity of the TCR (T cell Receptor) and thereby inhibits TCR-induced activation of the NFAT pathway by TCR (T cell receptor) activator molecule. This co-culture assay may be used to assay inhibitors of PDL1 binding to PD1 as these inhibitors will block the PD1-mediated inhibition of TCR-induced activation of the NFAT pathway.

Jurkat PD-1 reporter cells are pre-incubated with a PD-1 antagonist antibody (Ab) and then U2OS PD-L1/TCR activator cells are added to activate the TCR. The PD-1 Ab blocks PD-L1 activation of PD-1 and blocks PD-1 attenuation of the TCR activation and the final result is an increase in TCR activation with higher concentrations of PD-1 Ab.

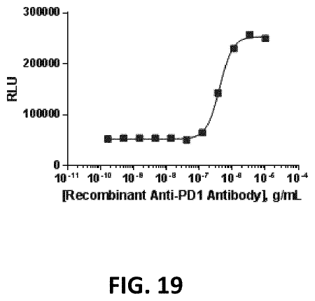

FIG. 19 shows that the Jurkat NFAT pathway reporter assay cell line can be used to produce PD-1 Pathway Reporter cell line demonstrating how a pathway reporter assay can be further modified to generate assays for other targets (other receptors and ligands).

### Example 12—Cell Line for U2OS NF-κB Pathway Reporter Assay

A reporter cell line was engineered to express a carrier protein-Enzyme Donor (ED) tagged carrier protein controlled by a pathway-inducible transcriptional response element. Pathway activation results in induced expressions of the carrier protein-ED-tagged protein. Addition of exogenous Enzyme Acceptor (EA), and buffer, lyses the cell and forces complementation of the ED and EA enzyme fragments. This results in the formation of a functional enzyme that hydrolyzes substrate to generate a chemiluminescent signal.

In this example, the cell line is a NFκB (nuclear factor NF-kappa-B p100 subunit) pathway reporter cell line. The cells are U2OS cells that include a nucleic acid encoding a reporter fragment and β-galactosidase donor fragment (ED) having the amino acid sequence NSLAVVLQRRDWENPGVTQLNRLAAHPPFASWRNSEEARTDR (SEQ ID NO:30) operably coupled to a promoter region comprising a NFκB response element. The cells also endogenously express CD40 (receptor).

Cells were plated in a 96-well plate and incubated at 37° C. and 5% CO₂ to allow the cells to attach and grow. Cells were then stimulated with a control agonist (here, CD40L), using the assay conditions described below. Following stimulation, signal was detected using the PathHunter® ProLabel®/ProLink™ Detection Kit (Eurofins DiscoverX Corporation) according to the recommended protocol.

### Assay Conditions

| Cell Plating Reagent | CP3 |
|---|---|
| Cell Number/Well | 5000 |
| Plate Type | 96 well |
| Cell Seeding Time (hours) | overnight |
| Control Agonist | CD40L |
| Ligand Incubation Time | 6 h |
| Ligand Incubation Temperature (° C.) | 37 |

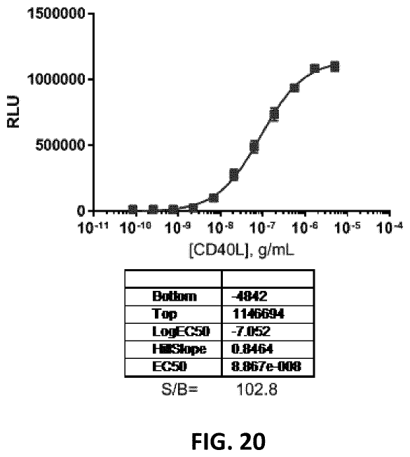

Results are shown in FIG. 20. This reporter cell line exhibited an EC₅₀ for control agonist stimulation of 0.0886 μg/mL and a signal:background ratio at 102.8

Other endogenous receptors and ligands that signal through the NFκB pathway have also be successfully used in this assay (e.g. TNFα through TNFR).

### Example 13—U2OS RANK-NFκB Pathway Reporter Assay

A reporter cell line was engineered to express a carrier protein-Enzyme Donor (ED) tagged carrier protein controlled by a pathway-inducible transcriptional response element. Pathway activation results in induced expressions of the carrier protein-ED-tagged protein. Addition of exogenous Enzyme Acceptor (EA), and buffer, lyses the cell and forces complementation of the ED and EA enzyme fragments. This results in the formation of a functional enzyme that hydrolyzes substrate to generate a chemiluminescent signal.

In this example, the cell line is a NFκB (Nuclear factor NF-kappa-B p100 subunit) pathway reporter cell line. The cells are U2OS cells that include a nucleic acid encoding a reporter fragment and β-galactosidase donor fragment (ED) having the amino acid sequence NSLAVVLQRRDWENPGVTQLNRLAAHPPFASWRNSEEARTDR (SEQ ID NO:30) operably coupled to a promoter region comprising a NFκB response element. To produce the RANK-NFκB reporter assay RANK (Receptor activator of nuclear factor κ B receptor) is co-expressed in the above-developed U2OS NFκB Pathway Reporter cells.

Cells were plated in a 96-well plate and incubated at 37° C. and 5% CO₂ to allow the cells to attach and grow. Cells were then stimulated with a control agonist (here, sRANKL), using the assay conditions described below. Following stimulation, signal was detected using the PathHunter® ProLabel®/ProLink™ Detection Kit (Eurofins DiscoverX Corporation) according to the recommended protocol.

### Assay Conditions

| Cell Plating Reagent | CP22 |
|---|---|
| Cell Number/Well | 5000 |
| Plate Type | 96 well |
| Cell Seeding Time (hours) | 4 |
| Control Agonist | soluble RANKL |
| Ligand Incubation Time | 16 h |
| Ligand Incubation Temperature (° C.) | 37 |

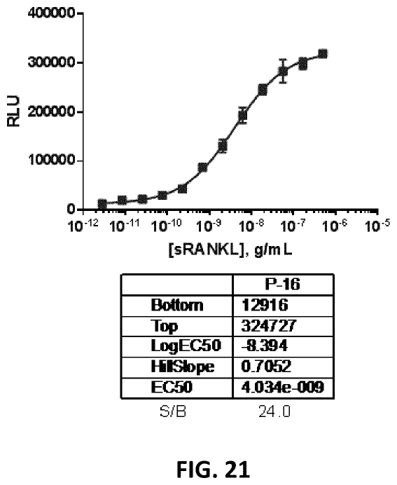

Results are shown in FIG. 21. This reporter cell line exhibited an EC₅₀ for control agonist stimulation of 4.034 ng/mL and a signal:background ratio at 24.0.

### Example 14—HEK NF-κB Pathway Reporter Assay

A reporter cell line was engineered to express a carrier protein-Enzyme Donor (ED) tagged carrier protein controlled by a pathway-inducible transcriptional response element. Pathway activation results in induced expressions of the carrier protein-ED-tagged protein. Addition of exogenous Enzyme Acceptor (EA), and buffer, lyses the cell and forces complementation of the ED and EA enzyme fragments. This results in the formation of a functional enzyme that hydrolyzes substrate to generate a chemiluminescent signal.

In this example, the cell line is a NF-κB (Nuclear factor NF-kappa-B p100 subunit) pathway reporter cell line. The cells are HEK-293 cells (HEK) that include a nucleic acid encoding a reporter fragment and β-galactosidase donor fragment (ED) having the amino acid sequence NSLAVVLQRRDWENPGVTQLNRLAAHPPFASWRNSEEARTDR (SEQ ID NO:30) operably coupled to a promoter region comprising a NFκB response element. Further, TNFα (ligand) and TNFR (receptor) expressed endogenously in HEK, were used to develop the NF-κB pathway reporter assay.

### Assay Conditions

| Cell Plating Reagent | CP3 |
|---|---|
| Cell Number/Well | 2500 |
| Plate Type | 384 well |
| Cell Seeding Time (hours) | overnight |
| Control Agonist | TNFα |
| Ligand Incubation Time | 6 h |
| Ligand Incubation Temperature (° C.) | 37 |

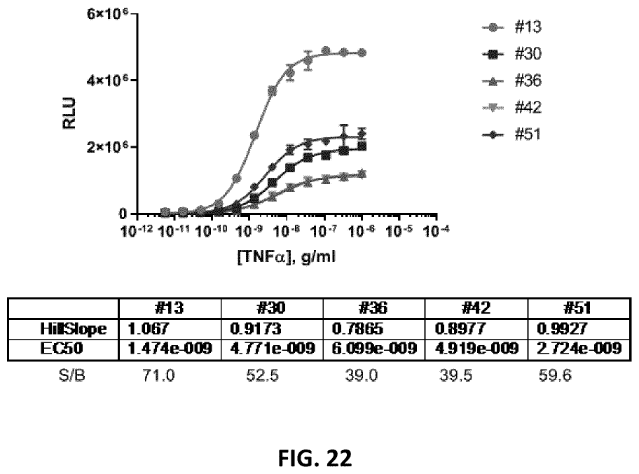

Results of the assay are shown in FIG. 22.

### Example 15—HEK CD27-NF-κB Pathway Reporter Assay

A reporter cell line was engineered to express a carrier protein-Enzyme Donor (ED) tagged carrier protein controlled by a pathway-inducible transcriptional response element. Pathway activation results in induced expressions of the carrier protein-ED-tagged protein. Addition of exogenous Enzyme Acceptor (EA), and buffer, lyses the cell and forces complementation of the ED and EA enzyme fragments. This results in the formation of a functional enzyme that hydrolyzes substrate to generate a chemiluminescent signal.

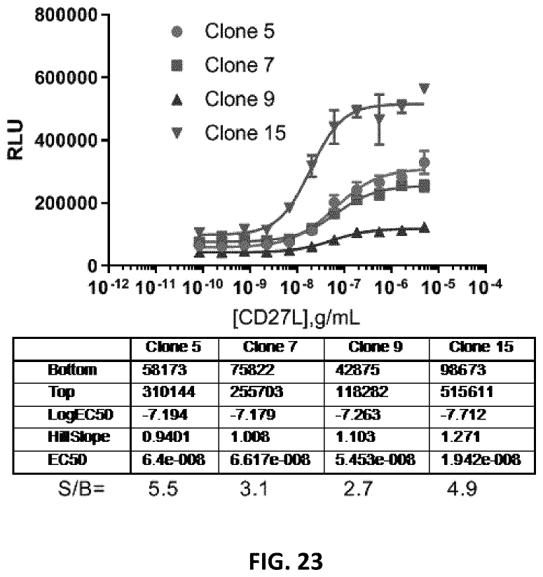

In this example, the cell line is a CD27-NF-κB pathway reporter cell line. The cells are HEK cells that include a nucleic acid encoding a reporter fragment and β-galactosidase donor fragment (ED) having the amino acid sequence NSLAVVLQRRDWENPGVTQLNRLAAHPPFASWRNSEEARTDR (SEQ ID NO:30) operably coupled to a promoter region comprising a NFκB response element. CD27 (receptor) is co-expressed in the above-developed HEK NF-κB pathway reporter cell line. Results of the assay are shown in FIG. 23.

### Assay Conditions

| Cell Plating Reagent | CP7 |
|---|---|
| Cell Number/Well | 2500 |
| Plate Type | 384 well |
| Cell Seeding Time (hours) | 4 |
| Control Agonist | CD27L |
| Ligand Incubation Time | 16 h |
| Ligand Incubation Temperature (° C.) | 37 |

### Example 16—Comparison of Assay Results from NF-κB Reporter Cell Line with Assay Results from RANK-NF-κB Reporter Cell Line

Two reporter cell lines were engineered to express a carrier protein-Enzyme Donor (ED) tagged carrier protein controlled by a pathway-inducible transcriptional response element. Pathway activation results in induced expressions of the carrier protein-ED-tagged protein. Addition of exogenous Enzyme Acceptor (EA), and buffer, lyses the cell and forces complementation of the ED and EA enzyme fragments. This results in the formation of a functional enzyme that hydrolyzes substrate to generate a chemiluminescent signal.

In this example, a first cell line is a U2OS-NF-κB pathway reporter cell line and the second cell line is U2OS RANK-NF-κB cell line. The cells are U2OS for both the cell lines as prepared in this example 16. The U2OS-NF-κB cell line includes a nucleic acid encoding a reporter fragment and β-galactosidase donor fragment (ED) having the amino acid sequence NSLAVVLQRRDWENPGVTQLNRLAAHPPFASWRNSEEARTDR (SEQ ID NO:30) operably coupled to a promoter region comprising a NFκB response element. The U2OS RANK-NF-κB cell line includes a nucleic acid encoding a reporter fragment and β-galactosidase donor fragment (ED) having the amino acid sequence NSLAVVLQRRDWENPGVTQLNRLAAHPPFASWRNSEEARTDR (SEQ ID NO:30) operably coupled to a promoter region comprising a NFκB response element followed by co-expression of RANK-CD27 (receptor) in the above-developed U2OS NF-κB pathway reporter cell line.

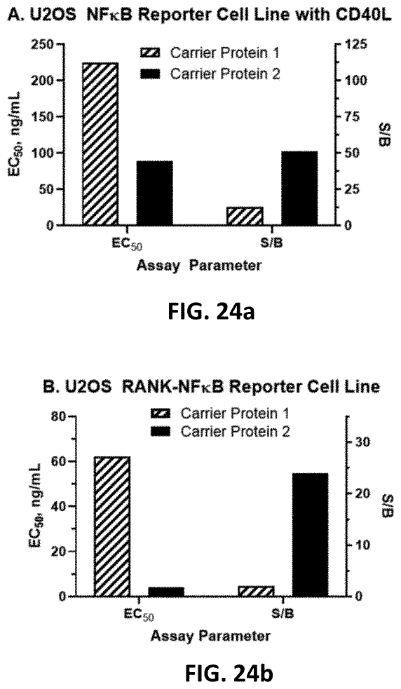

Assay results for U2OS NF-κB cell line with CD40L ligand is shown in FIG. 24a and assay results for U2OS RANK NF-κB cell line with sRANK ligand is shown in FIG. 24b. As shown in FIGS. 24a and 24b, the assay results for U2OS RANK NF-κB shows lower EC50 and a larger signal:background ratio.

The assay shows that RANK-NF-κB carrier protein shows better results that NF-κB carrier protein. Accordingly, one carrier protein may show a better result than another carrier protein.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of assessing activity of a promoter region, comprising:
culturing a cell comprising a nucleic acid, the nucleic acid comprising a region that encodes an enzyme donor (ED) operably coupled to a promoter region, under conditions in which the ED is expressed when the promoter region is active;
contacting the ED, if expressed, with an enzyme acceptor (EA) to form ED-EA complexes having enzymatic activity;
detecting the level of the enzymatic activity to assess activity of the promoter region; and
assessing the activation level of the transcription factor based on the detected level of the enzymatic activity.

2. The method according to claim, wherein the promoter region comprises a transcription factor response element (TFRE) for a transcription factor of interest, and wherein the activity of the promoter region is indicative of activity of the transcription factor.

3. (canceled)

4. The method according to claim 1, further comprising introducing into the cell an expression vector that encodes the transcription factor, and culturing the cell under conditions in which the transcription factor is expressed.

5. The method according to claim 1, further comprising contacting the cell with an agent, and assessing the activity level of the promoter region in response to contacting the cell with the agent based on the detected level of the enzymatic activity.

6. The method according to claim 5, wherein the activity of the promoter region is indicative of activity of a cell signaling pathway of interest.

7. The method according to claim 5, wherein contacting the cell with the agent comprises culturing the cell in the presence of the agent.

8. The method according to claim 5, wherein assessing the activity level of the promoter region in response to contacting the cell with the agent comprises comparing the level of enzymatic activity detected in the absence of the agent to the level of enzymatic activity detected in the presence of the agent.

9. The method according to claim 5, wherein the agent is a small molecule.

10. (canceled)

11. (canceled)

12. The method according to claim 1, wherein the nucleic acid further encodes a carrier protein fused to the ED, such that ED-carrier protein fusions are expressed when the promoter region is active.

13. The method according to claim 14, wherein the carrier protein exhibit an enzymatic activity which is not same as the ED-EA complex's enzyme activity.

14. (canceled)

15. (canceled)

16. A method of assessing activity of a promoter region, comprising:
culturing a cell comprising a nucleic acid, the nucleic acid comprising a region that encodes a carrier protein fused to an enzyme donor (ED) forming a ED-carrier protein fusion, wherein the ED-carrier protein fusion is operably coupled to a promoter region, under conditions in which the ED-carrier protein fusion is expressed when the promoter region is active;
contacting the ED, if expressed, with an enzyme acceptor (EA) to form ED-EA complexes having enzymatic activity;
detecting the level of the enzymatic activity to assess activity of the promoter region; and
assessing the activation level of the transcription factor based on the detected level of the enzymatic activity.

17. The method according to claim 16, wherein the promoter region comprises a transcription factor response element (TFRE) for a transcription factor of interest, and wherein the activity of the promoter region is indicative of activity of the transcription factor.

18. The method according to claim 17, wherein the carrier protein comprises a domain selected to affect the stability of the ED-carrier protein fusions.

19. The method according to claim 18, wherein the domain is selected to increase the stability of the ED-carrier protein fusions as compared to ED-carrier protein fusions lacking the domain.

20. The method according to claim 18, wherein the domain is selected to destabilize the ED-carrier protein fusions as compared to ED-carrier protein fusions lacking the domain.

21. The method according to claim 16, further comprising introducing into the cell an expression vector that encodes the transcription factor, and culturing the cell under conditions in which the transcription factor is expressed.

22. The method according to claim 16, comprising contacting the cell with an agent, and assessing the activity level of the promoter region in response to contacting the cell with the agent based on the detected level of the enzymatic activity.

23. The method according to claim 16, wherein the activity of the promoter region is indicative of activity of a cell signaling pathway of interest.

24. The method according to claim 22, wherein assessing the activity level of the promoter region in response to contacting the cell with the agent comprises comparing the level of enzymatic activity detected in the absence of the agent to the level of enzymatic activity detected in the presence of the agent.

25. The method according to claim 22, wherein the agent is a small molecule.

26. (canceled)

27. (canceled)

28. (canceled)


SEQUENCE LISTING (39 sequences)

SEQ ID NO: 1 - DNA, 126 bp, Artificial Sequence
  Synthetic nucleotide
aattcactggccgtcgttttacaacgtcgtgactgggaaaaccctggcgttacccaacttaatcgccttgcagcacatccccctttcgccagctggcgtaatagcgaagaggcccgcaccgatcgc

SEQ ID NO: 2 - DNA, 31 bp, Artificial Sequence
  Synthetic nucleotide
agagggtatataatggaagctcgacttccag

SEQ ID NO: 3 - DNA, 123 bp, Artificial Sequence
  Synthetic nucleotide
aattctcacggcttccctcccgaggtggaggagcaggccgccggcaccctgcccatgagctgcgcccaggagagcggcatggatagacaccctgctgcttgcgccagcgccaggatcaacgtc

SEQ ID NO: 4 - DNA, 48 bp, Artificial Sequence
  Synthetic nucleotide
gcttgcaagaactggttcagtagcttaagccactttgtgatccacctt

SEQ ID NO: 5 - DNA, 30 bp, Artificial Sequence
  Synthetic nucleotide
ggaggaaaaactgtttcatacagaaggcgt

SEQ ID NO: 6 - DNA, 52 bp, Artificial Sequence
  Synthetic nucleotide
gggaatttccggggactttccgggaatttccggggactttccgggaatttcc

SEQ ID NO: 7 - DNA, 14 bp, Artificial Sequence
  Synthetic nucleotide
ggtaagtaggtcat

SEQ ID NO: 8 - DNA, 25 bp, Artificial Sequence
  Synthetic nucleotide
agcctgatttccccgaaatgacggc

SEQ ID NO: 9 - DNA, 19 bp, Artificial Sequence
  Synthetic nucleotide
catttcccgtaaatcgtcg

SEQ ID NO: 10 - DNA, 15 bp, Artificial Sequence
  Synthetic nucleotide
agttctgagaaaagt

SEQ ID NO: 11 - DNA, 372 bp, Artificial Sequence
  Synthetic nucleotide
cttttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagttccctatcactctctttaatcactactcacagtaacctcaactcctgcca

SEQ ID NO: 12 - DNA, 22 bp, Artificial Sequence
  Synthetic nucleotide
acccccttaaagaaaggaggaa

SEQ ID NO: 13 - DNA, 30 bp, Artificial Sequence; identical to SEQ ID NO: 5
  Synthetic nucleotide

SEQ ID NO: 14 - DNA, 11 bp, Artificial Sequence
  Synthetic nucleotide
aattgcatgaa

SEQ ID NO: 15 - DNA, 11 bp, Artificial Sequence
  Synthetic nucleotide
gggatttcacc

SEQ ID NO: 16 - DNA, 21 bp, Artificial Sequence
  Synthetic nucleotide
atgaaggtaatgttttttcag

SEQ ID NO: 17 - DNA, 21 bp, Artificial Sequence
  Synthetic nucleotide
gtctttgaaaatatgtgtaat

SEQ ID NO: 18 - DNA, 10 bp, Artificial Sequence
  Synthetic nucleotide
aaacattttg

SEQ ID NO: 19 - DNA, 10 bp, Artificial Sequence
  Synthetic nucleotide
taatattttt

SEQ ID NO: 20 - DNA, 52 bp, Artificial Sequence
  Synthetic nucleotide
cagaattaacagtataaattgcatctcttgttcaagagttccctatcactct

SEQ ID NO: 21 - DNA, 452 bp, Artificial Sequence
  Synthetic nucleotide
actcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgctagcggaggaaaaactgtttcatacagaaggcgtggaggaaaaactgtttcatacagaaggcgtggaggaaaaactgtttcatacagaaggcgtagatctactagagggtatataatggaagctcgacttccagcttggcaatccggtactgttggtaaagccacc

SEQ ID NO: 22 - DNA, 653 bp, Artificial Sequence
  Synthetic nucleotide
actcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtaccttttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccagctag

SEQ ID NO: 23 - DNA, 449 bp, Artificial Sequence
  Synthetic nucleotide
actcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtaccagcctgatttccccgaaatgacggcagcctgatttccccgaaatgacggcagcctgatttccccgaaatgacggcagcctgatttccccgaaatgacggcagatctactagagggtatataatggaagctcgacttccagcttggcaatccggtactgttggtaaagccacc

SEQ ID NO: 24 - DNA, 494 bp, Artificial Sequence
  Synthetic nucleotide
actcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcagcttcatttcccgtaaatcgtcgaagcttcatttcccgtaaatcgtcgaagcttcatttcccgtaaatcgtcgaagcttcatttcccgtaaatcgtcgaagcttcatttcccgtaaatcgtcgactcgaggatatcaagatctactagagggtatataatggaagctcgacttccagcttggcaatccggtactgttggtaaagccacc

SEQ ID NO: 25 - DNA, 444 bp, Artificial Sequence
  Synthetic nucleotide
actcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcagttctgagaaaagtagttctgagaaaagtagttctgagaaaagtagttctgagaaaagtagttctgagaaaagtctcgaggatatcaagatctactagagggtatataatggaagctcgacttccagcttggcaatccggtactgttggtaaagccacc

SEQ ID NO: 26 - DNA, 445 bp, Artificial Sequence
  Synthetic nucleotide
actcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcggtaagtaggtcatggtaagtaggtcatggtaagtaggtcatggtaagtaggtcatggtaagtaggtcatcgtgacctcgaggatatcaagatctactagagggtatataatggaagctcgacttccagcttggcaatccggtactgttggtaaagccacc

SEQ ID NO: 27 - DNA, 467 bp, Artificial Sequence
  Synthetic nucleotide
actcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtaccaagatttctgcagcggagtactgtcctccgagcggagtactgtcctccgagcggagtactgtcctccgagcggagtactgtcctccgagcggagtactgtcctccgctcgaggatatcagatctactagagggtatataatggaagctcgacttccagcttggcaatccggtactgttggtaaagccacc

SEQ ID NO: 28 - protein, 40 aa, Artificial Sequence
  Synthetic polypeptide
SHGFPPEVEEQAAGTLPMSCAQESGMDRHPAACASARINV

SEQ ID NO: 29 - protein, 16 aa, Artificial Sequence
  Synthetic polypeptide
ACKNWFSSLSHFVIHL

SEQ ID NO: 30 - protein, 42 aa, Artificial Sequence
  Synthetic polypeptide
NSLAVVLQRRDWENPGVTQLNRLAAHPPFASWRNSEEARTDR

SEQ ID NO: 31 - protein, 52 aa, Artificial Sequence
  Synthetic polypeptide
MGVITDSLAVVLQRRDWENPGVTQLNRLAAHPPFASWRNSEEARTDRPSQQL

SEQ ID NO: 32 - protein, 52 aa, Artificial Sequence
  Synthetic polypeptide
MGVITDSLAVVLQRRDWENPGVTQLNRLAAHPPFASYRNSEEARTDRPSQQL

SEQ ID NO: 33 - DNA, 4120 bp, Artificial Sequence
  Synthetic nuleotide
aaattctcacggcttccctcccgaggtggaggagcaggccgccggcaccctgcccatgagctgcgcccaggagagcggcatggatagacaccctgctgcttgcgccagcgccaggatcaacgtcttcgaattgggaggtggcggtagcggaggtggcggtagcctcgagagctccaattcactggccgtcgttttacaacgtcgtgactgggaaaaccctggcgttacccaacttaatcgccttgcagcacatccccctttcgccagctggcgtaatagcgaagaggcccgcaccgatcgctagtgaggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaaactctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcat

SEQ ID NO: 34 - DNA, 3990 bp, Artificial Sequence
  Synthetic nucleotide
aagcttggaggtggcggtagcggaggtggcggtagcctcgagagctccaattcactggccgtcgttttacaacgtcgtgactgggaaaaccctggcgttacccaacttaatcgccttgcagcacatccccctttcgccagctggcgtaatagcgaagaggcccgcaccgatcgctagggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaaactctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcat

SEQ ID NO: 35 - DNA, 4119 bp, Artificial Sequence
  Synthetic nucleotide
aattctcacggcttccctcccgaggtggaggagcaggccgccggcaccctgcccatgagctgcgcccaggagagcggcatggatagacaccctgctgcttgcgccagcgccaggatcaacgtcttcgaattgggaggtggcggtagcggaggtggcggtagcctcgagagctccaattcactggccgtcgttttacaacgtcgtgactgggaaaaccctggcgttacccaacttaatcgccttgcagcacatccccctttcgccagctggcgtaatagcgaagaggcccgcaccgatcgctagtgaggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaaactctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcat

SEQ ID NO: 36 - DNA, 4120 bp, Artificial Sequence; identical to SEQ ID NO: 33
  Synthetic nucleotide

SEQ ID NO: 37 - DNA, 4120 bp, Artificial Sequence; identical to SEQ ID NO: 33
  Synthetic nucleotide

SEQ ID NO: 38 - DNA, 4120 bp, Artificial Sequence; identical to SEQ ID NO: 33
  Synthetic nucleotide

SEQ ID NO: 39 - DNA, 4120 bp, Artificial Sequence; identical to SEQ ID NO: 33
  Synthetic nucleotide